SEQUENCE LISTING (12 sequences)

SEQ ID NO: 1 - DNA, 1438 bp, synthetic construct
  source 1..1438: mol_type = other DNA
gagctcttgacagctagctcagtcctaggtataatactagtaccattaatttaataaaagggttttagagctagaaatagcaagttaaaataaggctagtccgttatcaacttgaaaaagtggcaccgagtcggtgctttttttgtcgactaaatgcagcaattgaagctgctagagcgggagaagcaggaaagggattttctgtggtggctgaagaaattggaaagcttgcagaggaatcaacatcagcaacaaaagaggttaaaaaacttcttgaagatataaaaagtaaaaatagtgtagtatttaaatcaatagatgtttctctaaaattatcagaggaacaaactgaatcagttaaagagactaaagaaatatttaataagatattagagtctttaaatagtttagtaggagaaatagaaaatataaggggctctataaatgacacttatcagagtaagaactttatatttggtaagttagaaagcatatcagcagcatcagaacaatcagcatctagttctgaggaagtatctgcaactacagaagaagtatcagctagtatgagtgaatttaataagatgtctcaaaaattagaacaagttgtagaggaacttgagagagaaatacaaaactttacgctgtaagctggcagaagtcactgaagcggccgactttttaacaaaatatattgataaaaataataatagtgggtataattaagttgttagagaaaacgtataaattagggataaactatggaacttatgaaatagattgaaatggtttatctgttaccccgtagggcccagaatttaaaaggagggattaaaatgcgtaaaaagtctttagcatttttgttagcactaacaatgttggtgacattattaggggctcagcttacagcttttgcagcaggtactggcgtcgttcaaatacaatttgctgatacaaatactagtacaaccatgaatactattgctcctaaatttaaaatcacaaataatactggagcacctttagatttaacaactttaaaattaagatactattttacagctgatggtactcaggatgaaaatttttggtgcgaccatgctggtatgcttaatggttataactaccaaacaattacaagtaatgtagtgggtacttttgtagctatggataatgcaacagctactgctgatcattatcttgagataagcttctcaaatggagcaggacaacttgatgcaggttcttcacttgaagttcaatgcagagttgcaaagaatgactggagtaattatgatcaatcaaacgattattcatttacttctaatgcaagtgattttactgactgggataagataacaggttatgttaatggagatcttgtattcggaaatccaccagtagtagacccagttattactccaactactgcgaattc

SEQ ID NO: 2 - DNA, 5029 bp, synthetic construct
  source 1..5029: mol_type = other DNA
gagctcccgcggtgcggccgcgggccctactttttaacaaaatatattgataaaaataataatagtgggtataattaagttgttactaaatgtaaaatgttagcgttttagagctagaaatagcaagttaaaataaggctagtccgttatcaacttgaaaaagtggcaccgagtcggtgcttttttgtcgacgcggccgctctagaactagtggatccttgccgaaaaagggatctgacagtaaatccatcaggtttcattgttgcagatgaaaatgataaggatgtagatcatgcatcaacagatggaaaaataacaatcacaggttcagcaccagttgttgtaaattcgtctgtaaatacttctagtgtaacttatgatccagcagcaccacaggatcaagctgtcagtgttacacttaatggcaatacaatcacagatgtaaaggacgcaaacgctagtgttcttaaggctggaagtgattacacagtaacaagtgatggaattacactaagccaaagctatcttgctactctagcagcaggaacttacacatatacagttgattttagtgcaggaaatgcaggtacatttactgttgttgttaagggaaaagcagtagtaaataaaactactttagcagtaggagctgcatcaggaaaagcaggagatactgttaaggtgcctgtaactataagtaaagtaacaacaccagtaggtttaatatgcatggaaatagattatgatgcaagtaagtttactgttaaggatgtacttcctaatacagatcttgtaaaagatactgataactacagctttattgctaatacgacatcagcaggaaaaatcagtattacatttacagatccaacacttgagaaattcccaataagtgcagatggagttatagcaaatatagattttgttgtaaattcaggtgcagcaactggtgatagcgatttaacagtaaattcatcaggtttcattgttgcagatgaaagtgatacagatatagatcatgtatcaacaaatggaaaaataactgttgaataatcaatgacataatactgccgccattataggatagcggcagtatactataaaattttaattaattatttttaaaggagatagaaaaatatgttaaagataagtaagaattttaaaaaaataatggctgtagctcttacatctacagttatatttggttctctatctggattattaactaataaagttgcagctgctacaactacagattcatccttaaaagtagataatgcgtatactcaaagatttgaaacaatgtacaatagaatgcacgatgctaataatggatatttcagtaaagatggtgttccatatcactcagttgaaacctttatggttgaggcacctgattatggtcatgaaactacaagtgaagctttcagttattatatgtggcttgaagcaatgcagggaaaaatcacaggaaactttagtggagtgaataccgcatgggatacagctgaaaagtatatgattccatcacaccaagatcaaccaggtatggatagatataacgctagtagtccagcaacatattcaccagaatgggaagatccaagtaagtatccatctagaatggatcaaggagcagcaaaaggacaagatccaataagtgatgagcttaaatcagcatatggaacttcagatatgtatggaatgcattggttaatggatgttgacaactggtatggctttggaaatcatgaagatggaacatctaaaaatgtttatataaacacttatcaaagaggagaacaggaatctgtttttgaaacagtacctcaaccatgttgggatgctttcaaatatggtggaaaaaatggatatcttgacttattcacaggagataatagctatgcaaaacaagctaaatacacagatgcaccagatgctgatgctcgtgcaatacaggcaacttatgaagcagcacaagcagctaaagaggatggagtagatttaagctcaatcgtaggtaaagcttctaaaatgggagattacttaagatatgctatgtttgataaatattttagaaaaataggaaattcaactcaagcaggaaatggtaaagattcaatgcattatttactttcttggtattatgcttggggtggatctcaaaataatgattggtcttggaaaataggctgcagtcacagtcattttggatatcaaaatcctttaactgcatgggtactttcaactgatagtcaattcaaacctaaatcagcaactggtgcaactgactgggcaaagagtttaacaactcaggtagatttctatcaatggttacaatcttcagagggagctatagcaggtggtgctagtaactcaaatcatggtcgttatgaagcatggccagaaggtacagctacatttgatggaatgggatatcaagaagaaccagtttaccatgatccaggtagtaacacatggtttggaatgcagtcatggtcaatgcagcgtatggctcaatactactatcaatcaaaagacccaaaagcaaaagctttacttgacaaatgggttaaatggattaaatctgtagttaaagtaaatccaaatggtgcaggaacatttgaagtaccatcaaaattaagttggactggacaaccagatacatggacaggatcttatacaggaaatcctaacttacatgtaaatgttgattcatatactactgatataggtacaagctcatcaactgcagatgcacttgcatactatgcagcagctacaggagataaggattcacaagcactttcaaaaactatacttgatgatatctggaaaaactatcaggatgcaaagggtgtatcagcaccagaacaaatggactatagccgtgtattcaatcaagaagtttatatcccacaaggttggacaggaacaatgcctaatggagatgtaattaaatcaggaaataaattcatagacatacgttcacaatataagaatgatcctgattatgctagagttaaatctgatgttgaagcaggaaagtcaagtacatttaactatcatcgtttctgggcagaaagtgagtatgctatagcaaatgcaaattatggaactttattcgctaatacagctacaccaggagatgtaaatggagacggcgtagttaatggaagagatcttatggagcttagacagtatttagcaggtaagttagatgctagtaaaataaatttagctgcagcagatgttaataatgatggtgtagttaatggaagagatattatggaacttactaaattaattgcaaaatagatcttataagaaaataacggaaggaagagtattcaatgaagaaaaaaggaataattacagcagtaatattgagtttactagttattggtacggtaggttgtaaatcaaacgacacaaagagtactgtatccgctaatcttgtaagcagtgaaaagtctaatttaaagataagcagtagtagcggaaaaacaggaagtaatgtggaggttaaagttaaggcaagtaatgtagctaaaaagaatgttatttgctgcgattttaaaataaaatatgatgctagtaaattagacgtttctggtatttcaccaggagaaattctaaaagatcctaaagataaccttgagtacaatgtggatagcaaaaatggtgttattacaattttatattcgtactcagataaaaatataggaaaagaattgatatcaaaaaatggggattttgtaactttaaacttgaaaataaaagatgatgctaaaaagggaaaaactcaaataagctttaatggcaatcctgaattctatgataagaatcaaaaaggtgtgtcagttactactaataaaggtgaaatagaaattaaataggccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttcttagtagaatcttaatttatgggagaagtgtttgatatgaaaaaaaaagttttgcttaagtgctatttttgcatcgttcatggcagcatgtcttgcatttggtaatgtagcctacgcagatgatgttaatgtaagtaattctaacgattatcttcacagtgatggaagtaagcttttagatgactatggtaatcaggttagaatgactggtatagcttggtttggacttgagactccaaattactgttttcatggcttgtgggctaataggcttgacaatattcttaatatagttgcagataatggatttaacactcttagagtgccattatctgttgaacttgtaaatcagtggcgtcaaggagtttatcctactccagattcaataaacgattatataagtcctgaattaaagggacaaaatagccttcaaatacttgatgatgtcatagcttatagtaaaaaagttggcgtcaaagttatgctagatatgcacagaattgaaagtggtggacagacagctacttggtatacaagtaaatatactacagatgactatgaaaaatgttggcaatatcttgctgatcgctataagaacgatgatacggttatagcagcagatatatttaacgagccacatggaaaagcttatagagcagaaacttctgctaagtggaatgatacaacagatgaagacaactggagatacgaagcagaaaaggttggtaaaaagattttagatataaatcctaagatgctcatagttgttgaaggagtagagacttaccctaaagagggaacagcagctggaagtacaaatcctgatgactactacggtggttggtggggaggaaatctaagaggtgttaaagattatcctgtagatttagccccatatgaattc

SEQ ID NO: 3 - DNA, 5048 bp, synthetic construct
  source 1..5048: mol_type = other DNA
gagctcccgcggtgcggccgcgggccctactttttaacaaaatatattgataaaaataataatagtgggtataattaagttgttactaaatgtaaaatgttagcgttttagagctagaaatagcaagttaaaataaggctagtccgttatcaacttgaaaaagtggcaccgagtcggtgcttttttgtcgacgcggccgctctagaactagtggatccttgccgaaaaagggatctgacagtaaatccatcaggtttcattgttgcagatgaaaatgataaggatgtagatcatgcatcaacagatggaaaaataacaatcacaggttcagcaccagttgttgtaaattcgtctgtaaatacttctagtgtaacttatgatccagcagcaccacaggatcaagctgtcagtgttacacttaatggcaatacaatcacagatgtaaaggacgcaaacgctagtgttcttaaggctggaagtgattacacagtaacaagtgatggaattacactaagccaaagctatcttgctactctagcagcaggaacttacacatatacagttgattttagtgcaggaaatgcaggtacatttactgttgttgttaagggaaaagcagtagtaaataaaactactttagcagtaggagctgcatcaggaaaagcaggagatactgttaaggtgcctgtaactataagtaaagtaacaacaccagtaggtttaatatgcatggaaatagattatgatgcaagtaagtttactgttaaggatgtacttcctaatacagatcttgtaaaagatactgataactacagctttattgctaatacgacatcagcaggaaaaatcagtattacatttacagatccaacacttgagaaattcccaataagtgcagatggagttatagcaaatatagattttgttgtaaattcaggtgcagcaactggtgatagcgatttaacagtaaattcatcaggtttcattgttgcagatgaaagtgatacagatatagatcatgtatcaacaaatggaaaaataactgttgaataatcaatgacataatactgccgccattataggatagcggcagtatactataaaattttaattaattatttttaaaggagatagaaaaatatgttaaagataagtaagaattttaaaaaaataatggctgtagctcttacatctacagttatatttggttctctatctggattattaactaataaagttgcagctgctacaactacagattcatccttaaaagtagataatgcgtatactcaaagatttgaaacaatgtacaatagaatgcacgatgctaataatggatatttcagtaaagatggtgttccatatcactcagttgaaacctttatggttgaggcacctgattatggtcatgaaactacaagtcaagctttcagttattatatgtggcttgaagcaatgcagggaaaaatcacaggaaactttagtggagtgaataccgcatgggatacagctgaaaagtatatgattccatcacaccaagatcaaccaggtatggatagatataacgctagtagtccagcaacatattcaccagaatgggaagatccaagtaagtatccatctagaatggatcaaggagcagcaaaaggacaagatccaataagtgatgagcttaaatcagcatatggaacttcagatatgtatggaatgcattggttaatggatgttgacaactggtatggctttggaaatcatgaagatggaacatctaaaaatgtttatataaacacttatcaaagaggagaacaggaatctgtttttgaaacagtacctcaaccatgttgggatgctttcaaatatggtggaaaaaatggatatcttgacttattcacaggagataatagctatgcaaaacaagctaaatacacagatgcaccagatgctgatgctcgtgcaatacaggcaacttatgaagcagcacaagcagctaaagaggatggagtagatttaagctcaatcgtaggtaaagcttctaaaatgggagattacttaagatatgctatgtttgataaatattttagaaaaataggaaattcaactcaagcaggaaatggtaaagattcaatgcattatttactttcttggtattatgcttggggtggatctcaaaataatgattggtcttggaaaataggctgcagtcacagtcattttggatatcaaaatcctttaactgcatgggtactttcaactgatagtcaattcaaacctaaatcagcaactggtgcaactgactgggcaaagagtttaacaactcaggtagatttctatcaatggttacaatcttcagagggagctatagcaggtggtgctagtaactcaaatcatggtcgttatgaagcatggccagaaggtacagctacatttgatggaatgggatatcaagaagaaccagtttaccatgatccaggtagtaacacatggtttggaatgcagtcatggtcaatgcagcgtatggctcaatactactatcaatcaaaagacccaaaagcaaaagctttacttgacaaatgggttaaatggattaaatctgtagttaaagtaaatccaaatggtgcaggaacatttgaagtaccatcaaaattaagttggactggacaaccagatacatggacaggatcttatacaggaaatcctaacttacatgtaaatgttgattcatatactactgatataggtacaagctcatcaactgcagatgcacttgcatactatgcagcagctacaggagataaggattcacaagcactttcaaaaactatacttgatgatatctggaaaaactatcaggatgcaaagggtgtatcagcaccagaacaaatggactatagccgtgtattcaatcaagaagtttatatcccacaaggttggacaggaacaatgcctaatggagatgtaattaaatcaggaaataaattcatagacatacgttcacaatataagaatgatcctgattatgctagagttaaatctgatgttgaagcaggaaagtcaagtacatttaactatcatcgtttctgggcagaaagtgagtatgctatagcaaatgcaaattatggaactttattcgctaatacagctacaccaggagatgtaaatggagacggcgtagttaatggaagagatcttatggagcttagacagtatttagcaggtaagttagatgctagtaaaataaatttagctgcagcagatgttaataatgatggtgtagttaatggaagagatattatggaacttactaaattaattgcaaaatagatcttataagaaaataacggaaggaagagtattcaatgaagaaaaaaggaataattacagcagtaatattgagtttactagttattggtacggtaggttgtaaatcaaacgacacaaagagtactgtatccgctaatcttgtaagcagtgaaaagtctaatttaaagataagcagtagtagcggaaaaacaggaagtaatgtggaggttaaagttaaggcaagtaatgtagctaaaaagaatgttatttgctgcgattttaaaataaaatatgatgctagtaaattagacgtttctggtatttcaccaggagaaattctaaaagatcctaaagataaccttgagtacaatgtggatagcaaaaatggtgttattacaattttatattcgtactcagataaaaatataggaaaagaattgatatcaaaaaatggggattttgtaactttaaacttgaaaataaaagatgatgctaaaaagggaaaaactcaaataagctttaatggcaatcctgaattctatgataagaatcaaaaaggtgtgtcagttactactaataaaggtgaaatagaaattaaataggccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttcttagtagaatcttaatttatgggagaagtgtttgatatgaaaaaaaaagttttgcttaagtgctatttttgcatcgttcatggcagcatgtcttgcatttggtaatgtagcctacgcagatgatgttaatgtaagtaattctaacgattatcttcacagtgatggaagtaagcttttagatgactatggtaatcaggttagaatgactggtatagcttggtttggacttgagactccaaattactgttttcatggcttgtgggctaataggcttgacaatattcttaatatagttgcagataatggatttaacactcttagagtgccattatctgttgaacttgtaaatcagtggcgtcaaggagtttatcctactccagattcaataaacgattatataagtcctgaattaaagggacaaaatagccttcaaatacttgatgatgtcatagcttatagtaaaaaagttggcgtcaaagttatgctagatatgcacagaattgaaagtggtggacagacagctacttggtatacaagtaaatatactacagatgactatgaaaaatgttggcaatatcttgctgatcgctataagaacgatgatacggttatagcagcagatatatttaacgagccacatggaaaagcttatagagcagaaacttctgctaagtggaatgatacaacagatgaagacaactggagatacgaagcagaaaaggttggtaaaaagattttagatataaatcctaagatgctcatagttgttgaaggagtagagacttaccctaaagagggaacagcagctggaagtacaaatcctgatgactactacggtggttggtggggaggaaatctaagaggtgttaaagattatcctgtagatttagccccatatcagatccgtgagcaaaagggaattc

SEQ ID NO: 4 - DNA, 2195 bp, synthetic construct
  source 1..2195: mol_type = other DNA
gagctcccgcggtgcggccgcgggccctactttttaacaaaatatattgataaaaataataatagtgggtataattaagttgttacatatatattgacaccaatgttttagagctagaaatagcaagttaaaataaggctagtccgttatcaacttgaaaaagtggcaccgagtcggtgcttttttgtcgacgcggccgctctagaactagtggatccaaagaagctggtttctttggattagttggtattgtgggtgctttggctgcacctgtagttgggaaagttgcagataaaaaaacaccaagatttgcagttggatttgctataatcttttctacaatagcttatgtttgtttttggggcatgggatataaaatatatggacttgtaattggagttattttgcttgatttaggaaatcaaaccggacaagtttcaaatcaagcaagagttcaagctataagtgatcaagagagaagtcgtataaatactgtatttatggtttcatattttattggaggttcaattggatcatttgttgcagctgtattttggcaaaagtttggatggagtggtgtgtgtgctattggtatgttttttcaaataattgcagttatatttcattattttatatacggaactaaatttagcattagaaataagtatggacaggtaaggtgatatataagtgtaaaattacaattttaaggttttagctatgaaaaagtagtctagaggcaggttgctttaaatataattttgtggtaaaatataactattaatataaagaatacctatttgtttggaaacaatgatgaatttctttaaattgggcacttgagaaattttgagttagtagtgcaaccgaccaacgattaattaagctgtattaattgttggttttttgcttgtgtaaggaggtgtttaaactattaaagtataaaaatgtaatttatgtattattgttcaatataatttacataatagggatataagatgtagagaggttaaataaaataatagccgactttttaacaaaatatattgataaaaataataatagtgggtataattaagttgttagagaaaacgtataaattagggataaactatggaacttatgaaatagattgaaatggtttatctgttaccccgtagggcccagaatttaaaaggagggattaaaatgttaaggagaaaattactgtctatgatagttgctgcttctcttgtagttggagtaggattctctaatatttgttatgcaaaaccaccagctccagatccaaacagcaatgtaggtacacatgatcttattagaaatagtacttttactgatggagttggattgccatggactgaggttgaaacagcacctgctcatggagattttgacatttcaggaggtacttataatataactgttacaaaaccaggaagtaatatctgggatgtacagtttagacatagaaatttacagcttaaagcaggacataaatatcatgtagaatttacagtaacagcagataaggattgtgatatttatcctcaaatagctatgtctaaagatccatatactcaatattggcactatggaaactgggaaaatgtacacttaacagcaggacaagctaaaactgtaactgatgatttcactatgacaacaaatgatgattctgctgaatttgcattccatatttccaatacaaacgataattcaaagcttccaataacatataaattcgataatatacatttaacagatccacaatatacacaaccagcaataccagatgataatatttacgatgcggtaagggtaaaccaagtcggatattatcctaatcttgagaagattgcaacagtaacttctgattcttcaactccaataccatggaaacttcaagacagcacaggagcagttgttgcatcaggacaaacaaaagtatttggacaagatcaggcatcaggagataatgttcatattatcgatttcacttcctacaataaatccggtaaggactataaattagtagttagtggtgatactgatgaaaatccagcatacagtgttccttttaatattggaagtgacttatattcacaacttaaacaagattctataaaatacttctatcacaacagaagtggtatagaaataaaaatgccttattgtggtgattcatggaattc

SEQ ID NO: 5 - DNA, 975 bp, synthetic construct
  source 1..975: mol_type = other DNA
ctcgggcccttgacagctagctcagtcctaggtataatactagtaatacaagtgatgataacgggttttagagctagaaatagcaagttaaaataaggctagtccgttatcaacttgaaaaagtggcaccgagtcggtgcttttttggatccatgttcttagtatattacctgtataacgattttctaagatttattttacgctccatattaacaaaaaatggattatacaactctaaattttatcataataatacattatttccaattgcaaacataatagttattcacaagaaaaacccctctttctgtaagatgtaaacggttatttttacattaatgcttttaacaagcttttttgcttatttaacaacagttgacactttttatttacaaatattataattattatatactaaattacaaatttaataaaataatgcacttagggggaatattttatgtaattctaggtataactaaaacactcttaccctattacaattatgtaatagggtatatctttctgaaatataatgctaattttgatattttcatttccacaatattttataactttatccatctagtatatacaaaattatcattacttttccaaatttttatttcatattacatcttatctacgaattacaatgcattatttaattttcatcatttgagacaattcttatatagtcatgctatataggtatataatggcaaactctgtactttaaagccgtgtaaacgtttcttttcacttgaatatcactatagtaacttttttactatttatcaattagttgacatgtaactttttaaataatataattatcacataattatgttatagatttaacaaaataacatacgaaaggaggaggcttttatgaaatgcaaattactttctactatacttaccacaatcattttatccactttaggaagctttaacaatgtgggcgccgaattc

SEQ ID NO: 6 - DNA, 1319 bp, synthetic construct
  source 1..1319: mol_type = other DNA
ggagctcttgacagctagctcagtcctaggtataatactagtatttctataagatatatgaagttttagagctagaaatagcaagttaaaataaggctagtccgttatcaacttgaaaaagtggcaccgagtcggtgcttttttggatccttatgaaggataaacgtttctctggttcaaaacgatgcataatgtagaagtagccgttttgtgcaagaaagagtgctatattttcatctataatagtttgcatatttgcaggtacaacaggcagtctaaagctatgctctcccaatattacacttgtatcgcattctgatcgactacgtacaatgcattttgcgggaatcagttgaatatcttcgtaatcaaatacactttccataggtacacctctatatataaattttaaggatagcttataatagtgtttgaaaatttatttatattatgcaaaattttagtgagattaatagtgaatgatgtagaaaagaagtatgtaataataataacttactaaaatgttaaagtatacagccttacttaaaaaagtataaagtaattactagaaatatttataaacgttagctttttagcgtttatcttaataaatagaaggttgtaagatacaacaagaattaatagaaattccgactttttaacaaaatatattgataaaaataataatagtgggtataattaagttgttagagaaaacgtataaattagggataaactatggaacttatgaaatagattgaaatggtttatctgttaccccgtagggcccagaatttaaaaggagggattaaaatgaggaacaaaaaacgaataacatcgttagtaactggtttagctatgttattcacttgtgctgttggaaatacttctcttaaagtacatgccgacgcacaatctatttacacaacgaaaggcgaaacgacaaagatttatgccagtgcttttacacaaaatactgatgactggacttggatgagtatgggggatactgctagtctagtgtatcaggatgtgacaaatttcaatgcggtagatgctaatagtgcatttgcaaagtcaaattctactgccaactttggtataaacatttcggacggaaaccttgcagagggggattcaagtacattaaaatttcatgttggtacggtcacagttaaagcaaatggctatgatgatcttgtaattaatttagataaagattattcagaagcgtatactgcaacaaaatcttcctgggggcttacaggaaatactactcaaattttactaaatgattatttgccaaaagacacgaattc

SEQ ID NO: 7 - DNA, 27 bp, synthetic construct
  source 1..27: mol_type = other DNA
agcggccgcaattaattattaaataaa

SEQ ID NO: 8 - DNA, 23 bp, synthetic construct
  source 1..23: mol_type = other DNA
gctagcaaagctaatgtgatgct

SEQ ID NO: 9 - DNA, 6687 bp, synthetic construct
  source 1..6687: mol_type = other DNA
caggataaaaaaattgtagataaattttataaaatagttttatctacaatttttttatcaggaaacagctatgaccgcggccatctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagatcgattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataaagcggccgcctttagagaggatgatactatgaaaaaattactcactgtaattcttatcttgacacttttatctattccttactctgtaaaatctgcgaaagcagaaactaatgtacgtgtcccagttcttctatatcatgttgtttctacaaatccagaccctaataatctttatcaatttagtcttacagaattcaaaaagcatatggattatctaaacgctaatggatatacgacactttctattgaccaatattacaatattataaacaaaaaggctcctatgcctaagaagccagttatgcttacctttgatgattgtactgaagacttctatacaaatgtatatcctattttaaggaaataccatatgaaagcagccgaatttgcaatcacaaatctaattgatacctatggacatttaactacaagtcagcttaaaactgttttctataacggaattgatgtagagaatcacactacaaatcacttagatttaactactttaacacataaccaaaagtatgctgcaatcaataatgcaactgccaaaattaagtctataaccaataaagctccactttacttggcatacccttatggaacatatgatgcagatagtgtttcaatccttaaaagtttaggttataaagctggtttttccgtatcaaacgtcttaagcaccgacacaagtaacaaatatggtttacctcgtattgttattacaaatggcgataccttaaatgtatttgaaaaaaagcttttaaatggtcattaaaattaattattaaataaaaaaaggagtgttattatgttaaaatcaaaattatcaaaaatatgtacaggagtcttagctttaggtcttgccctttcaatttcaggtgtaggaacttttaaagctgctatgtcacatagcaaatttgtaggaaatattatagcaggaagtattccttctaactttgatacctattggaatcaagttacaccagaaaatgcaactaagtggggcgcaattgaatatggtcgtggcaattataactggggaagcgcagatcttatttataattacgccagaagtaaaaacatgccattcaaatttcataatttagtatggggaagtcagcagcctacttggttgtcaaatctttcacctcaagatcaaaaatctgaagtatcaaaatggattgcagccgcaggtcaaagatattctggttcagcttttgttgatgttgtaaatgaaccactgcatactcaaccttcttacaaaaatgctttaggcggagatggttccaccggttatgattggattgtatggtcttatcagcaggcaagaaaagccttccctaattcaaaacttttaattaatgaatatggcataataggcgatcctaatgcagcagctaattatgttaaaatcataaatgttcttaaaagcaaaggtttaattgatggaataggaattcaatgtcactatttcaatatggataacgtttctgtaggaacaatgaactatgttttaaatatgttatctaatacaggtttaccaatatacgtatcagaacttgatatgactggcgatgactcaactcagcttgctagatatcaacaaaagttccctgttctatatcaaaatcctaatgtaaaaggtataactttatggggatatatgcaaggtcaaacttggaatagtggtacttatttagttaattcaaatggtactgaacgtccagctcttaaatggttaagatcttacttagcatcacattagctttgctagcaaagtattgttaaaaataactctgtagaattataaattagttctacagagttattttttaaaaaaattctaaacttatgtataaaaaatacgataagaatgtagaattaaaactaaagacagttcaatttcttttagaataatttagttagtgtggtaaaaaaatgtcataatgatatttatgttgaaatttgtataaaattcagaaaatgaatatattttatcaattttcagtcatttgaaagattatgaggctaatgcagtactaggcgtaaattgaatttataattactatagcgataagaaatggcctaaaaacgtttgcagtaatgaaagaaccgtaaatattataaaaaaaatcttaaaacagagttttatttataaaaatttaagatatataatttaaataacgtgttaaaatagtggaggaagtaatttgaatctgaatattaaaagaatgttaaaggttgtaactctttatgatgcaattattgctgcaatagtttcagtaatacttttgtttgctgctaattataagatttcgttaatagtgattatagggattttttcagcaatatttaatttttatttaagtaatttaacagctgatttcgtttttgtaaaaaaaatgggaaatacgtcacttatatttcttagttcaatttttagagtaatacttgttttttttataggtattattctttataaaatatataaatattatttaatagcctacttaggaggatatagtgctcattttatagcccttataatttatgggtcactagtaaataaacgatgaaaggaagtgattgaatggagctaggtgcaaagacagtattttcgatgaagcttggaagttacaactttgctataacagaaactgtagtattacagtggattatcatggcagttataatattacttgcaatatttcttactaaaaatcttaagaaagtaccaaataggaaacaaagcgtaatagaaatgattgttaacttaataaatggattggtaaaagaaaatatgggagagaaattcatgaatttcgttccaattatcggtactatggcagtgtttatacttttcttaaatttaacagggctagtaggtatcgaaccagcaacaaaggatattagtgttacagcaggctttgctttagtaagtgcatttttaataaatgcaactgcaataaaaagaaggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctg

SEQ ID NO: 10 - DNA, 11163 bp, synthetic construct
  source 1..11163: mol_type = other DNA
ctagcaaagtattgttaaaaataactctgtagaattataaattagttctacagagttattttttaaaaaaattctaaacttatgtataaaaaatacgataagaatgtagaattaaaactaaagacagttcaatttcttttagaataatttagttagtgtggtaaaaaaatgtcataatgatatttatgttgaaatttgtataaaattcagaaaatgaatatattttatcaattttcagtcatttgaaagattatgaggctaatgcagtactaggcgtaaattgaatttataattactatagcgataagaaatggcctaaaaacgtttgcagtaatgaaagaaccgtaaatattataaaaaaaatcttaaaacagagttttatttataaaaatttaagatatataatttaaataacgtgttaaaatagtggaggaagtaatttgaatctgaatattaaaagaatgttaaaggttgtaactctttatgatgcaattattgctgcaatagtttcagtaatacttttgtttgctgctaattataagatttcgttaatagtgattatagggattttttcagcaatatttaatttttatttaagtaatttaacagctgatttcgtttttgtaaaaaaaatgggaaatacgtcacttatatttcttagttcaatttttagagtaatacttgttttttttataggtattattctttataaaatatataaatattatttaatagcctacttaggaggatatagtgctcattttatagcccttataatttatgggtcactagtaaataaacgatgaaaggaagtgattgaatggagctaggtgcaaagacagtattttcgatgaagcttggaagttacaactttgctataacagaaactgtagtattacagtggattatcatggcagttataatattacttgcaatatttcttactaaaaatcttaagaaagtaccaaataggaaacaaagcgtaatagaaatgattgttaacttaataaatggattggtaaaagaaaatatgggagagaaattcatgaatttcgttccaattatcggtactatggcagtgtttatacttttcttaaatttaacagggctagtaggtatcgaaccagcaacaaaggatattagtgttacagcaggctttgctttagtaagtgcatttttaataaatgcaactgcaataaaaagaaggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcaggataaaaaaattgtagataaattttataaaatagttttatctacaatttttttatcaggaaacagctatgaccgcggccatctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagatcgattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataaagcggccgcggcccagaatttaaaaggagggattaaaatgcgtaaaaagtctttagcatttttgttagcactaacaatgttggtgacattattaggggctcagcttacagcttttgcagcaggtactggcgtcgttcaaatacaatttgctgatacaaatactagtacaaccatgaatactattgctcctaaatttaaaatcacaaataatactggagcacctttagatttaacaactttaaaattaagatactattttacagctgatggtactcaggatgaaaatttttggtgcgaccatgctggtatgcttaatggttataactaccaaacaattacaagtaatgtagtgggtacttttgtagctatggataatgcaacagctactgctgatcattatcttgagataagcttctcaaatggagcaggacaacttgatgcaggttcttcacttgaagttcaatgcagagttgcaaagaatgactggagtaattatgatcaatcaaacgattattcatttacttctaatgcaagtgattttactgactgggataagataacaggttatgttaatggagatcttgtattcggaaatccaccagtagtagacccagttattactccaactactgctacatttgatacagctaatccaactgatgtaaatgtagctatgcaattaaacggatttacattatcaggattaacagatgagaacggaacagctgttgatgctgcaaattatacaatatcaggaagtaacttggtattaagcaaggcttatcttgcaaaattagcattaggcaaacatagctttaattttaattttgctaaaggcactacaactataagtaaaccattagcacttacagttacagatacagcaggtataacagtagatcctgcaagtgtagtatttgataaagtagcacctcaagatgagaatgttgctcttaaattaaatggacatacacttggtgatgtggtaggacctaaaggaaaccttgtaaaaggaacagattatactgtagcagatgatggaactgtaacatttagtaaagcatatctttcaactcttccactaggtgatcagacaataacatttaaagctagtgatgattctactaaaacagcagcagtaacagttacagtgaagaattcaaatgctacaaatgttactgttggagatgttacaggagcaaaaaaaggagatactataaaggttcctgtaagtgtaagtacagtaaaaacaccaataggattaatagatatggaaataaactatgatccaacagagttaactgcaaaagatgtagttcctacagatttagtaaaggatactgataactatagctttatagtaaatacatcaactccaggtaagattagtattacatttacagatccaactcttggaacttatccaataggtacagatggaatatttgcatatttagaattccttgtagctggtgaaaaagcaggaaaatatgatttgaaagtaaacccaactacattaatacttgcagatgaaaatgataatgatatagattgtaacccaccaaaggatggaagtgtaactgttataggtactccagttgtaactccatctcaaataaatgttgaacaaggttcagcaactgatcagccagttaaaatagatttaaatggtaacacacttaaagatgttgttgatcaaagtggtaagactcttgttcaaggaacggattatacagtaacagatactggaatcacattaagtcaatcttatcttgcaggtttagccttaggtcaatacactctaacacttgattttaatggtggaggagcatcacagacaataactattaatgttgtaaagaatgaaactgtaaaattgtcagttggaacagtatcaggaaatccaggggatactgtaaaggtgcctgtaactataagccaggtatcaacaccagtaggtttaatatgtatggatataagctacgatgcaagtaagtttactgttaaggatgtacttcctaatacagatcttgtaaaggatactgataactacagctttatagtaaatacatcaactccaggtaagattagtattacatttacagatccaacacttgcaaactatccaataagtgtagacggaattcttgcatacttagactttattataaattcaaatgcaacagcaggagatagtgctttaacagtagatccagctacattaatagttgcagatgaaaatgataaagatataaaagatgcagcttctaatggaaaaataacagttacaggttcagcaccagttgttcaaagctcagtagtaaatacttcaagtgtaacttatgatcaaaatgcaccacaggatcaagctgttagtattactttcaatggcaatacagttaaggatgtaaaggacgcaagtggcaatacacttaaggctggaagtgactacacagcaacaagtgatggaattacacttagccaaagttatcttgctactttagcagcaggaacttacacatatacaattgattttagtgcaggaaatgcagggacatttactgttgttgttaaaggaaaaacagtagtaggtagtgcaactactttagcagtaggaactgtatcaggaaaagcaggagatactgtaaaggtacctgtaactatcagtaaagtaacaacaccagtaggtttaatatgtgcagaaatagactatgatgcaagcaagtttactgttaaggatgtacttcctaatacagatcttgtaaaggatactgataactacagctttatagtaaatacatcaactccaggtaagattagtattacatttacagatccaacacttgcaaactatccaataagtgcagatggaattcttgcatacttagactttatcataaactcaaatgcaacagcaggggatagtgctttaacagtaaatccatcaggatttatcattgcagatgaaaatgataaagatatacaggatgcagcttctaacggaaaaataacagttacaggttcaacaccagttgctgaaaattcagtagtaaatacttcaagtgtaacttatgatcaaaatgcaccacaggatcaagctgttagcattaccttaaatggtaatacaattacagatgtaaaagatgcaagtggtaatacacttaaggctggaagtgattacacagtaacaagtgatggaattacacttagccaaagctaccttgctactttagcagcaggaacttacacatatacagttgattttagtgcaggaaatgcaggaacatttactgttgttgttaaagcaaaaacagtagtaagtagtgcaactactttagcagtaggaacagtatcaggaaaagcaggagatactgtaaaggtacctgtaactataagtaaagtaacaacaccagtagggttaatatgtgcagaaatagattatgatgcaagcaagtttactgttaaggatgtacttcctaatacagatcttgtaaaagatactgataactacagctttatagtaaatactgcaacagcaggaaaaatcagtattacattcacagatccaacacttgagaaattcccaataagtgcagatggaattcttgcatacttagactttatcataaactcaaatgcaacagcaggggatagtgctttaacagtaaatccatcaggtttcattgttgcagatgaaaatgataaggatgtagatcatgcatcaacagatggaaaaataacaatcacaggttcagcaccagttgttgtaaattcgtctgtaaatacttctagtgtaacttatgatccagcagcaccacaggatcaagctgtcagtgttacacttaatggcaatacaatcacagatgtaaaggacgcaaacgctagtgttcttaaggctggaagtgattacacagtaacaagtgatggaattacactaagccaaagctatcttgctactctagcagcaggaacttacacatatacagttgattttagtgcaggaaatgcaggtacatttactgttgttgttaagggaaaagcagtagtaaataaaactactttagcagtaggagctgcatcaggaaaagcaggagatactgttaaggtgcctgtaactataagtaaagtaacaacaccagtaggtttaatatgcatggaaatagattatgatgcaagtaagtttactgttaaggatgtacttcctaatacagatcttgtaaaagatactgataactacagctttattgctaatacgacatcagcaggaaaaatcagtattacatttacagatccaacacttgagaaattcccaataagtgcagatggagttatagcaaatatagattttgttgtaaattcaggtgcagcaactggtgatagcgatttaacagtaaattcatcaggtttcattgttgcagatgaaagtgatacagatatagatcatgtatcaacaaatggaaaaataactgttgaataagcggccgcctttagagaggatgatactatgaaaaaattactcactgtaattcttatcttgacacttttatctattccttactctgtaaaatctgcgaaagcagaaactaatgtacgtgtcccagttcttctatatcatgttgtttctacaaatccagaccctaataatctttatcaatttagtcttacagaattcaaaaagcatatggattatctaaacgctaatggatatacgacactttctattgaccaatattacaatattataaacaaaaaggctcctatgcctaagaagccagttatgcttacctttgatgattgtactgaagacttctatacaaatgtatatcctattttaaggaaataccatatgaaagcagccgaatttgcaatcacaaatctaattgatacctatggacatttaactacaagtcagcttaaaactgttttctataacggaattgatgtagagaatcacactacaaatcacttagatttaactactttaacacataaccaaaagtatgctgcaatcaataatgcaactgccaaaattaagtctataaccaataaagctccactttacttggcatacccttatggaacatatgatgcagatagtgtttcaatccttaaaagtttaggttataaagctggtttttccgtatcaaacgtcttaagcaccgacacaagtaacaaatatggtttacctcgtattgttattacaaatggcgataccttaaatgtatttgaaaaaaagcttttaaatggtcattaaaattaattattaaataaaaaaaggagtgttattatgttaaaatcaaaattatcaaaaatatgtacaggagtcttagctttaggtcttgccctttcaatttcaggtgtaggaacttttaaagctgctatgtcacatagcaaatttgtaggaaatattatagcaggaagtattccttctaactttgatacctattggaatcaagttacaccagaaaatgcaactaagtggggcgcaattgaatatggtcgtggcaattataactggggaagcgcagatcttatttataattacgccagaagtaaaaacatgccattcaaatttcataatttagtatggggaagtcagcagcctacttggttgtcaaatctttcacctcaagatcaaaaatctgaagtatcaaaatggattgcagccgcaggtcaaagatattctggttcagcttttgttgatgttgtaaatgaaccactgcatactcaaccttcttacaaaaatgctttaggcggagatggttccaccggttatgattggattgtatggtcttatcagcaggcaagaaaagccttccctaattcaaaacttttaattaatgaatatggcataataggcgatcctaatgcagcagctaattatgttaaaatcataaatgttcttaaaagcaaaggtttaattgatggaataggaattcaatgtcactatttcaatatggataacgtttctgtaggaacaatgaactatgttttaaatatgttatctaatacaggtttaccaatatacgtatcagaacttgatatgactggcgatgactcaactcagcttgctagatatcaacaaaagttccctgttctatatcaaaatcctaatgtaaaaggtataactttatggggatatatgcaaggtcaaacttggaatagtggtacttatttagttaattcaaatggtactgaacgtccagctcttaaatggttaagatcttacttagcatcacattagctttg

SEQ ID NO: 11 - DNA, 36 bp, synthetic construct
  source 1..36: mol_type = other DNA
gcagatggcggccgcggcccagaatttaaaaggagg

SEQ ID NO: 12 - DNA, 42 bp, synthetic construct
  source 1..42: mol_type = other DNA
caacaaatggaaaaataactgttgaataagcggccgcatttc

(19) United States
(12) Patent Application Publication
(10) Pub. No.: US 2026/0085278 A1
(43) Pub. Date: Mar. 26, 2026

(54) GENETICALLY MODIFIED LIGNOCELLULOLYTIC CLOSTRIDIUM ACETOBUTYLICUM

(71) Applicants: Institut National des Sciences Appliquees de Toulouse, Toulouse Cedex 4 (FR); Institut National de Recherche Pour I'Agriculture, I'Alimentation et I'Environnement, Paris (FR); Centre National de la Recherche Scientifique (CNRS), Paris (FR)

(72) Inventors: Tom WILDING-STEELE, Toulouse Cedex 4 (FR); Quentin RAMETTE, Toulouse Cedex 4 (FR); Philippe SOUCAILLE, Toulouse Cedex 4 (FR)

(73) Assignees: Institut National des Sciences Appliquees de Toulouse, Toulouse Cedex 4 (FR); Institut National de Recherche Pour I'Agriculture, I'Alimentation et I'Environnement, Paris (FR); Centre National de la Recherche Scientifique (CNRS), Paris (FR)

(21) Appl. No.: 19/110,846

(22) PCT Filed: Sep. 15, 2023
(86) PCT No.: PCT/EP2023/075525

§ 371 Date: Mar. 12, 2025

(30) Foreign Application Priority Data
Sep. 15, 2022 (EP) 22306357.9

(51) Int. Cl. C12N 1/20; C12N 9/24; C12N 9/42; C12P 7/06; C12P 7/16; C12P 7/56; C12R 1/145

(52) U.S. Cl. CPC C12N 1/20; C12N 9/2437; C12N 9/2482; C12P 7/06; C12P 7/16; C12P 7/56; C12R 2001/145; C12Y 302/01004; C12Y 302/01008

(57) ABSTRACT

The present invention relates to a genetically modified Clostridium acetobutylicum able to grow on lignocellulosic biomass, in which the cip-cel operon is overexpressed, the expression level of each gene of the cip-cel operon in the genetically modified Clostridium acetobutylicum being higher than its expression level in a corresponding non-genetically modified Clostridium acetobutylicum, as well as derivatives thereof with further modifications increasing growth on lignocellulosic biomass, and the use of these strains for the production of bulk chemicals by conversion of lignocellulose as source of carbon, such as ethanol, butanol, glycerol, 1,2-propanediol, acetone, isopropanol, isobutene, hydrogen, acetic acid and lactic acid and the like.

### TECHNICAL FIELD OF THE INVENTION

The present invention is in the field of bioproduction. It relates to a genetically modified strain of Clostridium acetobutylicum, which is able to grow on lignocellulosic biomass, due to higher expression level of the cip-cel operon compared to its expression level in a corresponding non genetically modified Clostridium acetobutylicum. It further relates to the use of the genetically modified strain of Clostridium acetobutylicum for the production of bulk chemicals by conversion of lignocellulose as source of carbon, such as ethanol, butanol, glycerol, 1,2-propanediol, 1,3-propanediol, acetone, isopropanol, hydrogen, acetic acid and lactic acid and the like.

### BACKGROUND ART

Clostridium acetobutylicum (abbreviated as C. acetobutylicum) is known for nearly 100 years to produce solvents such as butanol, as well as other bulk chemicals. However, although Clostridium acetobutylicum can utilize all the sugars resulting from cellulose hydrolysis (Compere and Griffith 1979) and hemicellulose (Dunning and Lathrop 1945; Sticheblykina and Nakhamanovich, B. M 1959) and degrades polymers such as starch or xylan, it is not able to hydrolyze and grow on crystalline cellulose or pretreated lignocellulosic biomass (Lee, Forsberg, and Gibbins 1985).

Cellulosome, a macromolecular complex for cellulose degradation (Bayer E. A., Y. Shoham, and R. Lamed. 2000), has been genetically and biochemically characterized in multiple Clostridium species including Ruminiclostridium cellulolyticum (Gal, L & al. 1997), Clostridium cellulovorans (Doi, R. H. & al. 1994), Clostridium josui (Kakiuchi, M. & al. 1998) and Clostridium thermocellum (Lamed, R., and E. A. Bayer. 1988). Surprisingly, the genome sequencing of C. acetobutylicum ATCC 824 (Nölling et al. 2001) has revealed the presence of a large cellulosomal gene cluster. Sequence analysis revealed that this cluster contains the genes for the CipA scaffolding protein, the Cel48A cellulase, several cellulases of family 5 and 9, the Man5G mannanase, and a hydrophobic protein, OrfXp. Genetic organization of this large cluster is very similar to those of the mesophilic R. cellulolyticum and C. cellulovorans (Belaïch, J. P. & al. 1997; Tamaru, Y. & al. 2000).

As C. acetobutylicum is unable to grow on cellulosic substrates, the existence of a cellulosomal gene cluster in the genome raises questions about its expression, function and evolution. In an earlier work, we have established that C. acetobutylicum can produce a cellulosome with an apparent molecular weight of about 665 kDa (Sabathé, F. & al. 2002). Biochemical and immunochemical analyses of the cellulosomal components revealed the existence of at least four subunits including the CipA scaffolding protein and three major cellulases, Cel48A, Cel9X, and Cel9C cellulases.

According to EP2436698A1, the cellulases Cel9X and Cel9C of C. acetobutylicum are active on crystalline cellulose, while Cel48A of C. acetobutylicum would be inactive, thus explaining the inability of C. acetobutylicum to grow on crystalline cellulose or pretreated lignocellulosic biomass. As a solution to this problem, EP2436698A1 provided a genetically modified C. acetobutylicum with a cel48SAFA hybrid gene (composed of the cel48A gene, in which the sequence encoding the catalytic domain is replaced by the catalytic domain of the cel48F gene).

However, even when replacing the native promoter of the cip-cel operon by a strong promoter, the expression level of the cel48SAFA hybrid gene remains relatively low, and the ability of a C. acetobutylicum to grow on cellulose is thus limited.

Tao et al (TAO XUANYU ET AL: “Precise promoter integration improves cellulose bioconversion and thermotolerance in Clostridium cellulolyticum”, METABOLIC ENGINEERING, vol. 60, 1 Jul. 2020 (2020-07-01), pages 110-118, ISSN: 1096-7176, DOI: 10.1016/j.ymben.2020.03.013) discloses engineered Ruminiclostridium cellulolyticum (abbreviated as “R. cellulolyticum”, the new name of Clostridium cellulolyticum, as evidenced by Yutin N et al. “A genomic update on clostridial phylogeny: Gram-negative spore formers and other misplaced clostridia”. Genomics update. Vol. 15, Issue 10, October 2013, pages 2631-2641, https://doi.org/10.1111/1462-2920.12173), in which synthetic P4 promoter or predicted P2 promoter was inserted between orfX and cel9H genes of the cip-cel gene cluster, resulting in increased expression of downstream genes in the cip-cel gene cluster, i.e. genes cel9J, cel9M, and cel5N, but not upstream genes such as cel48F. This resulted in enhanced cellulolytic activity.

Contrary to R. cellulolyticum, C. acetobutylicum and other related saccharolytic clostridia however, are able to utilise sugars at a very high catabolic rate (Mickaël Desvaux, Clostridium cellulolyticum: model organism of mesophilic cellulolytic clostridia, FEMS Microbiology Reviews, Volume 29, Issue 4, September 2005, Pages 741-764, https://doi.org/10.1016/j.femsre.2004.11.003), making them much better candidates as a consolidated bioprocessing (CBP) organism.

However, Ruminiclostridium cellulolyticum (the model species used in Tao et al) and C. acetobutylicum belong to two distinct major evolutionary groups of cellulosomal mesophilic bacteria, group one containing R. cellulolyticum (the species used by Tao et al), Clostridium sp. BNL1100, C. papyrosolvens and C. josui.while group two contains C. acetobutylicum, C. cellulovorans, C. bornimense, and C. saccharoperbutylacetonicum. In group two, only C. cellulovorans is able to utilise cellulose. The two distinct major evolutionary groups of cellulosomal mesophilic bacteria differ by their architectures of the cip-cel operon, their main scaffoldin gene, and regulation of the cip-cel operon by distinct promoters (see Dassa, Bareket, Ilya Borovok, Vincent Lombard, Bernard Henrissat, Raphael Lamed, Edward A. Bayer, and Sarah Moraïs. 2017. “Pan-Cellulosomics of Mesophilic Clostridia: Variations on a Theme” Microorganisms 5, no. 4: 74. https://doi.org/10.3390/microorganisms5040074).

In view of such evolutionary and structural differences, a skilled person would not have expected that using the same approach with C. acetobutylicum would also permit enhanced cellulolytic activity.

Even more importantly, Sabathé, F. et al. 2002 described C. acetobutylicum's cellulosome as being inactive and EP2436698A1 discloses Cel48A of C. acetobutylicum as inactive. Increasing the expression of a complex which has been demonstrated to be inactive, of which Cel48A is a major component, would clearly not be expected to create cellulolytic activity, as Tao et al only show a slight increase of the rate of growth on cellulose of a bacterium already able to grow on cellulose.

Finally, Mingardon et al. (Mingardon F. et al. “The Issue of Secretion in Heterologous Expression of Clostridium cellulolyticum Cellulase-Encoding Genes in Clostridium acetobutylicum ATCC 824”. APPLIED AND ENVIRONMENTAL MICROBIOLOGY, May 2011, p. 2831-2838. doi:10.1128/AEM.03012-10) showed that overexpressing the cel48A gene in C. acetobutylicum was toxic for the bacteria, further suggesting that using an approach similar to that disclosed in Tao et al would not succeed in C. acetobutylicum.

There is thus still a need for improved strains of C. acetobutylicum able to grow efficiently on crystalline cellulose or pretreated lignocellulosic biomass.

### SUMMARY OF THE INVENTION

In the context of the present invention, the inventors surprisingly found that, contrary to the teaching of EP2436698A1, the cel48A gene of C. acetobutylicum encodes a functional Cel48A protein that is in fact active on crystalline cellulose. In addition, the inventors found that overexpression of the whole cip-cel operon (with native genes but a replacement of their common promoter by the promoter of the thlA gene, a strong constitutive promoter, as in strain SC1 disclosed in Example 1) or at least cipA, cel48A and orfxp (as in strain SC2 disclosed in Example 1) resulted in the production of an active cellulosome, the resultant strain being able to grow on lignocellulosic biomass. They also found that, when under the control of the strong constitutive promoter of the thlA gene, native Cel48A protein is produced at significantly higher levels compared to hybrid Cel48SAFA protein. The ability to grow on lignocellulosic biomass could be further improved by overexpression of the cel9X gene and by overexpression of the cel5Y gene and/or the xynB gene, and/or by deletion of the extracellular protease encoding gene nrpE. The ability to grow on lignocellulosic biomass could also be further improved by increasing the ratio of the CipA scaffolding protein to cellulases of the cellulosome by increasing the amount of CipA being produced (as in strain SC10 comprising an additional copy of cipA gene under the control of the promoter of the thlA gene, disclosed in Example 10), this permitting to obtain a higher amount of cellulosome and a lower amount of free cellulases. This also allowed efficient growth on crystalline cellulose.

In a first aspect, the present invention thus relates to a genetically modified Clostridium acetobutylicum able to grow on lignocellulosic biomass, in which the cip-cel operon is overexpressed, the expression level of each gene of the cip-cel operon in the genetically modified C. acetobutylicum being higher than its expression level in a corresponding non-genetically modified C. acetobutylicum.

The present invention also relates to a method for the production of a targeted bulk chemical n lignocellulosic biomass, comprising culturing a genetically modified Clostridium acetobutylicum according to the invention on an appropriate culture medium comprising lignocellulosic biomass as main source of carbon, and recovering the targeted bulk chemical from the culture medium.

### DESCRIPTION OF THE FIGURES

FIG. 1. SDS-PAGE of the CipA:Cel48A and CipA:Cel48A_E61Q complex purified by cellulose affinity chromatography from C. acetobutylicum strains SC2 and SC3, respectively. M: molecular-weight marker. E61Q: CipA:Cel48A_E61Q. Cel48A: CipA:Cel48A.

FIG. 2. SDS_PAGE of the 30-fold concentrated supernatant from strain SC1 and strain Pthl-SAFA. M: molecular weight marker.

FIG. 3. SDS-PAGE of the cellulosomes purified from strains SC1 and SC4.

FIG. 4. A+B) Showing the rate of consumption and products produced for strain SC4 growing on either PASC or AECC. C) Comparison of the rate of consumption of AECC for strains SC1 and SC4.

FIG. 5. Comparison of the rate of consumption of AECC for strains SC4 and SC5.

FIG. 6. SDS-PAGE of the cellulosomes purified from strains SC4 and SC6. M: molecular weight marker.

FIG. 7. Comparison of the rate of consumption of AECC for strains SC4 and SC6.

FIG. 8. Comparison of the rate of consumption of AECC for strains SC4 and SC7.

FIG. 9. SDS-PAGE of the concentrated supernatant and purified cellulosome from strains SC4 and SC6.

FIG. 10. SDS-PAGE of the concentrated supernatant from strains SC9 and SC10

FIG. 11. SDS-PAGE of the purified cellulosomes from strains SC9 and SC10.

FIG. 12. Comparison of the rate of consumption of AECC for strains SC8, SC9 and SC10 FIG. 13. Comparison of the rate of consumption of AECC for strain SC10 and C. cellulovorans.

FIG. 14. Comparison of the rate of consumption of crystalline cellulose for stains SC1, SC9 and SC10.

### DETAILED DESCRIPTION OF THE INVENTION

### General Definitions

The term “bulk chemical(s)” means large volume chemicals.

“Clostridium acetobutylicum” or “C. acetobutylicum” is known in the art and means a bacterial strain of the Clostridium genus producing acetone, butanol and ethanol and possessing a megaplasmid carrying the solvent forming genes (Nölling et al, 2001). A “genetically modified Clostridium acetobutylicum” refers to a Clostridium acetobutylicum whose genome has been altered in the laboratory using genetic engineering techniques in order to favor the expression of desired physiological traits.

When referring to a genetically modified Clostridium acetobutylicum according to the invention with an overexpressed operon or gene, a “corresponding non-genetically modified Clostridium acetobutylicum” refers to a Clostridium acetobutylicum strain with a genome identical to that of the genetically modified Clostridium acetobutylicum according to the invention, excepted for the genetic modification inserted in the genome of the genetically modified Clostridium acetobutylicum according to the invention in order to overexpress said operon or gene.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. “Consisting essentially of” means excluding other components or steps of any essential significance. “Consisting of” means excluding more than trace elements of other components or steps.

A “constitutive promoter” refers to an unregulated promoter that allows for continual transcription of its associated gene or operon. Conversely, an “inducible promoter” refers to a regulated promoter that is active only in response to specific stimuli.

An “operon” refers to a functioning unit of DNA containing a cluster of genes under the control of a single promoter.

A “gene” is said to be “overexpressed” in a genetically modified Clostridium acetobutylicum when its expression level in the genetically modified Clostridium acetobutylicum is higher than the expression level of the cip-cel operon in the corresponding non-genetically modified Clostridium acetobutylicum.

An “operon” is said to be “overexpressed” in a genetically modified Clostridium acetobutylicum when at least one gene and preferably each gene of the operon has a higher expression level in the genetically modified Clostridium acetobutylicum than in a corresponding non-genetically modified Clostridium acetobutylicum.

A native promoter of a gene or operon is said to be “replaced” by another promoter when the nucleic acid sequence of the native promoter is removed from the Clostridium acetobutylicum genome and the nucleic acid sequence of another promoter is inserted in the Clostridium acetobutylicum genome and operably linked to the gene or operon, so that it may direct its expression.

A native gene is said to be “replaced” by another gene when at least the protein-encoding nucleic acid sequence of the gene is replaced by the protein-encoding nucleic acid sequence of another gene. The regulatory sequences of the native gene (promoter notably) may or not be replaced by those of the other gene.

Except otherwise indicated, all herein recited NCBI Reference Sequences (NCBI RefSeq) or Genbank accession numbers include the version number (for RefSeq or Genbank accession number X, version n will be written X.n), so that the precise sequence is fully defined. Annotations of the sequence are those as of Sep. 16, 2022.

Genetically Modified Clostridium acetobutylicum Able to Grow on Lignocellulosic Biomass

The present invention first relates to a genetically modified Clostridium acetobutylicum able to grow on lignocellulosic biomass, in which the cip-cel operon is overexpressed, the expression level of each gene of the cip-cel operon in the genetically modified Clostridium acetobutylicum being higher than its expression level in a corresponding non-genetically modified Clostridium acetobutylicum.

Clostridium acetobutylicum

Preferred strains of Clostridium acetobutylicum strains that may be genetically modified in the context of the invention are those known for their use (with or without genetic modifications) for the production of solvents and other bulk chemicals, such as Clostridium acetobutylicum ATCC 824, Clostridium acetobutylicum DSM 1731, and Clostridium acetobutylicum ATCC 4259, in particular Clostridium acetobutylicum ATCC 824, which has been used in the experimental part.

The genome of Clostridium acetobutylicum ATCC 824 has been sequenced and annotated and is publicly available under NCBI reference sequence NC_003030.1. The genome of Clostridium acetobutylicum DSM 1731 has been sequenced and annotated and is publicly available under NCBI reference sequence NC_015687.1.

### Ability to Grow on Lignocellulosic Biomass

The genetically modified Clostridium acetobutylicum according to the invention is able to grow on lignocellulosic biomass.

### Lignocellulosic Biomass

“Lignocellulosic biomass” means any carbon source which consists primarily of cellulose, hemicellulose and lignin. Lignocellulosic biomass is the most abundantly available raw material on the Earth for the production of biofuels such as ethanol or butanol, and the provision of bacteria feeding and able to produce such biofuels on lignocellulosic biomass is thus highly desirable.

Before fermentation with the genetically modified Clostridium acetobutylicum according to the invention, lignocellulosic biomass is preferentially pre-treated by one of many pre-established methods, including steam explosion, ammonia fiber expansion, alkali extraction (e.g. alkali extracted deshelled corn cobs, also referred to as “AECC”, a model lignocellulosic biomass which has been commonly used (Sudha Rani, Swamy, and Seenayya 1998, 22)), organosolv treatment, or sulfite treatment (Zhao et al. 2022).

However, crystalline cellulose may also be used. As used herein, “crystalline cellulose” refers to cellulose type I, which is native cellulose found in most plant cell walls and structures, and is composed of glucose units connected by a 1-4 beta glycosidic bonds. Crystalline cellulose notably includes microcrystalline cellulose (abbreviated as “MCC”), such as the commercially available Avicel PH products. An organism is generally considered a truly cellulolytic when it can be shown to be able to utilize crystalline cellulose as a carbon source and to degrade it substantially (see e.g., Koeck et al. Genomics of cellulolytic bacteria. Curr Opin Biotechnol. 2014. 29:171-83).

Crystalline cellulose may notably be used as a substrate when a genetically modified Clostridium acetobutylicum in which at least the native promoter of the cip-cel operon has been replaced by the promoter of Clostridium acetobutylicum thlA gene, the native promoter of the cel9X gene has been replaced by the promoter of Clostridium acetobutylicum thlA gene, and the native promoter of the cel5Y gene has been replaced by the promoter of Clostridium acetobutylicum thlA gene, an additional copy of the xynB gene has been inserted in the Clostridium acetobutylicum genome under the control of the promoter of Clostridium acetobutylicum thlA gene, an additional copy of the cipA gene has been inserted in the Clostridium acetobutylicum genome under the control of the promoter of Clostridium acetobutylicum thlA gene and the nrpE gene has been partially or completely deleted (such as the SC10 strain) is used. Strain SC10 can therefore be considered a true cellulolytic organism.

### Ability to Grow on Lignocellulosic Biomass

The ability to grow on lignocellulosic biomass may be measured and quantified in vitro using an appropriate culture medium comprising lignocellulosic biomass. For instance, a genetically modified Clostridium acetobutylicum is cultured on an appropriate culture medium comprising lignocellulosic biomass and the amount of lignocellulosic biomass consumed in the culture medium is monitored during at least 3 days. Optionally, the amount of one or more catabolite(s) produced from lignocellulosic biomass (e.g. butyrate, acetate, butanol, ethanol . . . depending on the specific tested strain) may also be monitored in the culture medium. If consumption of the lignocellulosic biomass of interest is detected (and optionally production of at least one catabolite is detected), then the Clostridium acetobutylicum is able to grow lignocellulosic biomass.

The expression “appropriate culture medium” refers to a culture medium adapted for the used genetically modified Clostridium acetobutylicum according to the invention, as it is well known by the man skilled in the art. Known appropriate culture media for cultivation of Clostridium acetobutylicum include, without limitation, the Clostridial Growth Medium (CGM) medium (Dusseaux et al. 2013), 2XYT (Oultram et al. 1988), Minimal synthetic (MS, Soni et al. 1987), and Clostridial Basal Medium (CBM, see O′BRIEN et al. 1971).

For measuring ability to grow on lignocellulosic biomass, the lignocellulosic biomass has preferably been pre-treated by one of many pre-established methods disclosed above.

### Overexpression of the Cip-Cel Operon

The genetically modified Clostridium acetobutylicum according to the invention has been genetically modified so that the cip-cel operon is overexpressed, i.e. its expression level is higher than the expression level of the cip-cel operon in a corresponding non-genetically modified Clostridium acetobutylicum.

### Cip-Cel Operon

Clostridium acetobutylicum “cip-cel operon” or “cip-cel cluster” refers to Clostridium acetobutylicum operon comprising the genes in Table 1 below:

TABLE 1
Description of genes included in the cip-cel operon.

| | Name in | | Name in | | Sequence of |
|---|---|---|---|---|---|
| | NC_003030.1 | | NC_015687.1 | | encoded protein |
| Conventional | (new/old | Position in | (new/old | Position in | (NCBI accession |
| name | locus tag) | NC_003030.1 | locus tag) | NC_015687.1 | number) |
| cipA | CA_RS04850/ | 1043161 to | SMB_RS04790/ | 1042982 to | WP_010964228.1 |
| | CA_C0910 | 1047612 | SMB_G0927 | 1047433 | |
| cel48A | CA_RS04855/ | 1047700 to | SMB_RS04795/ | 1047521 to | WP_010964229.1 |
| | CA_C0911 | 1049880 | SMB_G0928 | 1049701 | |
| cel5B | CA_RS04860/ | 1049962 to | SMB_RS04800/ | 1049783 to | WP_010964230.1 |
| | CA_C0912 | 1051410 | SMB_G0929 | 1051231 | |
| cel9C | CA_RS04865/ | 1051531 to | SMB_RS04805/ | 1051352 to | WP_010964231.1 |
| | CA_C0913 | 1053669 | SMB_G0930 | 1053490 | |
| orfXp | CA_RS04870/ | 1053705 to | SMB_RS04810/ | 1053526 to | WP_010964232.1 |
| | CA_C0914 | 1054250 | SMB_G0931 | 1054071 | |
| cel5D | CA_RS04875/ | 1054263 . . . | SMB_RS04815/ | 1054084 to | WP_010964233.1 |
| | CA_C0915 | 1056083 | SMB_G0932 | 1055904 | |
| cel9E | CA_RS04880/ | 1056114 . . . | SMB_RS04820/ | 1055935 to | WP_010964234.1 |
| | CA_C0916 | 1058192 | SMB_G0933 | 1058013 | |
| cel9F | CA_RS04885/ | 1058249 . . . | SMB_RS04825/ | 1058070 to | WP_010964235.1 |
| | CA_C0917 | 1059862 | SMB_G0934 | 1059683 | |
| man5G | CA_RS04890/ | 1059880 . . . | SMB_RS04830/ | 1059701 to | WP_010964236.1 |
| | CA_C0918 | 1061157 | SMB_G0935 | 1060978 | |
| cel9H | CA_RS04895/ | 1061241 . . . | SMB_RS04835/ | 1061062 to | WP_010964237.1 |
| | CA_C0919 | 1063760 | SMB_G0936 | 1063581 | |

The nucleic acid sequence of the entire cip-cel operon, without the promoter region, corresponds to positions 1043161 to 1063760 of NC_003030.1.

All the genes of the cip-cel operon display linked transcription, being under the control of the same promoter, which corresponds to positions 1040257 to 1043160 of NC_003030.1.

In the context of the present invention, the cel48A gene preferably encodes the native Cel48A protein of Clostridium acetobutylicum, i.e the protein of amino acid sequence WP_010964229.1. Preferably, the genetically modified Clostridium acetobutylicum comprises the native cel48A gene of nucleic acid sequence corresponding to positions 1047700 to 1049880 of NC_003030.1. In an embodiment, all the genes of the cip-cel operon are not genetically modified. In this case, the nucleic acid sequences of the genes of the cip-cel operon are those indicated in Table 1 above.

### Overexpression of the Cip-Cel Operon

The overexpression of the cip-cel operon is due to a genetic modification of the Clostridium acetobutylicum.

In order to overexpress the cip-cel operon, several types of genetic modifications may be made. In particular, the genetically modified Clostridium acetobutylicum according to the invention with an overexpressed cip-cel operon may be such that:
a) the native promoter of the cip-cel operon has been genetically modified, or
b) an additional copy of the cip-cel operon has been inserted in the Clostridium acetobutylicum genome.

In a preferred embodiment, as all the genes of the cip-cel operon are under the control of the same promoter, this promoter can be genetically modified in order to increase expression of the genes of the operon. Here also, several genetic modifications of the native promoter of the cip-cel operon may be used:
a1) the native promoter of the cip-cel operon may be replaced by a stronger promoter, or
a2) the native promoter of the cip-cel operon may be mutated.

The nucleic acid sequence of the native promoter of the cip-cel operon in Clostridium acetobutylicum is known in the art, corresponding to positions 1040257 to 1043160 of NC_003030.1.

Strong bacterial promoters are known in the art and option a1) above, in which the native promoter of the cip-cel operon may be replaced by a stronger promoter is a preferred embodiment.

A “stronger promoter” refers to a promoter able to induce a higher expression level of a given operon or gene than the native promoter. The stronger promoter is preferably constitutive, although a strong inducible promoter may also be used, provided that the promoter may be induced so that the cip-cel operon is expressed when the cells grow on lignocellulosic biomass. Examples of constitutive promoters stronger than the native promoter of Clostridium acetobutylicum cip-cel operon include, without limitation, the promoters of Clostridium acetobutylicum thlA gene, ptb gene, crt gene and gapC gene. The nucleic acid sequences of the above-mentioned genes and their promoters are known in the art, as described in Table 2 below.

TABLE 2
Relevant information regarding strong promoters.

| | Name in | | | Name in | | |
|---|---|---|---|---|---|---|
| Gene | NC_003030.1 | Position of | Position of | NC_015687.1 | | Position of |
| conventional | (new/old | the gene in | the promoter | (new/old | Position in | the promoter |
| name | locus tag) | NC_003030.1 | in NC_003030.1 | locus tag) | NC_015687.1 | in NC_015687.1 |
| thlA | CA_RS14790/ | 3005963 to | 3007176 to | SMB_RS14730/ | 3007590 to | 3008769 to |
| | CA_C2873 | 3007141 | 3007288 | SMB_G2909 | 3008768 | 3009000 |
| ptb | CA_RS15805/ | 3226192 to | 3227098 to | SMB_RS15740/ | 3227819 to | 3228725 to |
| | CA_C3076 | 3227097 | 3227205 | SMB_G3112 | 3228724 | 3228912 |
| crt | CA_RS13980/ | 2835666 to | 2836452 to | SMB_RS13920/ | 2837291 to | 2838077 to |
| | CA_C2712 | 2836451 | 2836626 | SMB_G2747 | 2838076 | 2838446 |
| gapC | CA_RS03825 | 821221 to | 821076 to | SMB_RS03780/ | 821202 to | 820922 to |
| | to CA_C0709 | 822225 | 821220 | SMB_G0723 | 822206 | 821201 |

In a particularly preferred embodiment, the native promoter of the cip-cel operon has been replaced by the promoter of Clostridium acetobutylicum thlA gene.

Replacement of the native promoter of the cip-cel operon by a stronger promoter may be performed using conventional genetic techniques, including replacement techniques efficient in Clostridia based on homologous recombination as disclosed in WO2008040387, ACE or CRISPR/Cas9 editing of Clostridium acetobutylicum genome (see Ehsaan et al. 2016 for ACE editing and Wilding-Steele et al. 2021 for CRISPR/Cas9 editing). Detailed technical information regarding one manner to perform the replacement is provided in Example 1 below.

However, the native promoter may also be kept in a mutated form resulting in higher expression of the cip-cel operon. In particular, retrocontrol elements present in the promoter, such as a catabolite-Responsive Element (CRE), may be deleted from the native promoter.

In this respect, it should be noted that the cip-cel operon of Clostridium acetobutylicum has been found to be highly similar to the cip-cel operon of Clostridium cellulolyticum (see Sabathé, F. & al. 2002), and the promoter of the cip-cel operon of Clostridium cellulolyticum has been studied, and a CRE was found in the promoter, the deletion of which resulted in much higher expression of the cip-cel operon (see Abdou et al, 2007). Similar genetic modifications of the native promoter of the cip-cel operon of Clostridium acetobutylicum may be contemplated in the context of the invention.

Alternatively, or in combination with a genetic modification of the native promoter of the cip-cel operon of Clostridium acetobutylicum, an additional copy of the cip-cel operon may be inserted in the Clostridium acetobutylicum genome.

In this case, the additional copy of the cip-cel operon is preferably put under the control of a promoter stronger than the native promoter of the cip-cel operon of Clostridium acetobutylicum (see above for exemplary stronger promoters).

Insertion of an additional copy of the cip-cel operon in the Clostridium acetobutylicum genome may be performed by conventional genetic techniques, including use of ACE as disclosed in Ehsaan et al. 2016 or CRISPR/Cas9 editing of Clostridium acetobutylicum genome (see Wilding-Steele et al. 2021).

The additional copy of the cip-cel operon inserted into the Clostridium acetobutylicum genome may or not contain a promoter stronger than the native promoter of the cip-cel operon operably linked to the cip-cel operon, depending on where the additional copy is inserted. When the additional copy of the cip-cel operon is inserted in a position where it is under the control of a native promoter of the Clostridium acetobutylicum genome, no promoter is necessary in the inserted sequence (see Example 8 below for the xynB gene). If not, then the inserted sequence should further comprise a promoter stronger than the native promoter of the cip-cel operon operably linked to the cip-cel operon.

### Optional Overexpression of Other Genes

In order to further improve growth on lignocellulosic biomass, the genetically modified Clostridium acetobutylicum according to the invention may be further genetically modified in order to overexpress other genes improving cellulosome activity.

Such genes improving cellulosome activity may notably be selected from cellulases located outside of the cip-cel operon and xylanases.

In particular, the genetically modified Clostridium acetobutylicum according to the invention may have been further genetically modified so that one or more of the genes of Table 3 below is overexpressed, its expression level in the genetically modified Clostridium acetobutylicum being higher than its expression level in a corresponding non-genetically modified Clostridium acetobutylicum.

TABLE 3
Clostridium acetobutylicum genes that may be further overexpressed in the genetically
modified Clostridium acetobutylicum according to the invention.

| | Name in | | | Name in | | |
|---|---|---|---|---|---|---|
| Gene | NC_003030.1 | Position of | Position of | NC_015687.1 | | Position of |
| conventional | (new/old | the gene in | the promoter | (new/old | Position in | the promoter |
| name | locus tag) | NC_003030.1 | in NC_003030.1 | locus tag) | NC_015687.1 | in NC_015687.1 |
| cel9X | CA_RS03075/ | 649182 to | 648365 to | SMB_RS03040/ | 649168 to | 648351 to |
| | CA_C0561 | 651818 | 649181 | SMB_G0574 | 651804 | 649167 |
| cel5Y | CA_RS17835/ | 3667802 to | 3670841to | SMB_RS17765/ | 3669384 to | 3669211 to |
| | CA_C3469 | 3670840 | 3671007 | SMB_G3507 | 3672422 | 3669383 |
| | Name in | | | Name in | | |
| Gene | NC_001988.2 | Position of | Position of | NC_015686.1 | | Position of |
| conventional | (new/old | the gene in | the promoter | (new/old | Position in | the promoter |
| name | locus tag) | NC_001988.2 | in NC_001988.2 | locus tag) | NC_015686.1 | in NC_015686.1 |
| xynB | CA_RS19485/ | 53626 to | 55366 to | SMB_RS19440/ | 53626 to | 55365 to |
| | CA_P0053 | 54582 | 55588 | SMB_P052 | 54582 | 55580 |

### Optional Overexpression of the Cel9X Gene

In a preferred embodiment, the genetically modified Clostridium acetobutylicum according to the invention has been further genetically modified so that the cel9X gene is overexpressed, its expression level in the genetically modified Clostridium 10 acetobutylicum being higher than its expression level in a corresponding non-genetically modified Clostridium acetobutylicum.

The overexpression of the cel9X gene is due to a genetic modification of the Clostridium acetobutylicum.

In order to overexpress the cel9X gene, several types of genetic modifications may be made. In particular, the genetically modified Clostridium acetobutylicum according to the invention with an overexpressed cel9X gene may be such that:
a) the native promoter of the cel9X gene has been genetically modified, or
b) an additional copy of the cel9X gene has been inserted in the Clostridium acetobutylicum genome.

In an embodiment, the native promoter of the cel9X gene can be genetically modified in order to increase expression of the cel9X gene. Here also, several genetic modifications of the native promoter of the cel9X gene may be used:
a1) the native promoter of the cel9X gene may be replaced by a stronger promoter, or
a2) the native promoter of the cel9X gene may be mutated.

The stronger promoter is preferably constitutive. Examples of constitutive promoters stronger than the native promoter of Clostridium acetobutylicum cel9X gene include, without limitation, the promoter of Clostridium acetobutylicum thlA gene, ptb gene, crt gene and the gapC gene as presented in Table 2 above.

Replacement of the native promoter of the cel9X gene by a stronger promoter may be performed using conventional genetic techniques (see above for the cip-cel operon and Example 3 below for a specific protocol).

Insertion of an additional copy of the cel9X gene in the Clostridium acetobutylicum genome may be performed by conventional genetic techniques (see general description above for the cip-cel operon and Examples 8 below for the xynB gene).

### Optional Overexpression of the Cel5Y Gene

Alternatively or in combination with further overexpression of the cel9X gene, the genetically modified Clostridium acetobutylicum according to the invention may have been further genetically modified so that the cel5Y gene is overexpressed, its expression level in the genetically modified Clostridium acetobutylicum being higher than its expression level in a corresponding non-genetically modified Clostridium acetobutylicum.

The overexpression of the cel5Y gene is due to a genetic modification of the Clostridium acetobutylicum.

In order to overexpress the cel5Y gene, several types of genetic modifications may be made. In particular, the genetically modified Clostridium acetobutylicum according to the invention with an overexpressed cel5Y gene may be such that:
a) the native promoter of the cel5Y gene has been genetically modified, preferably:
   a1) the native promoter of the cel5Y gene has been replaced by a stronger promoter, or
   a2) the native promoter of the cel5Y gene has been mutated.
b) an additional copy of the cel5Y gene has been inserted in the Clostridium acetobutylicum genome under the control of a promoter stronger than the native promoter of the cel5Y gene,

The stronger promoter is preferably constitutive. Examples of constitutive promoters stronger than the native promoter of Clostridium acetobutylicum cel5Y gene include, without limitation, the promoter of Clostridium acetobutylicum thlA gene, ptb gene, crt gene and the gapC gene, as presented in Table 2 above.

Replacement of the native promoter of the cel5Y gene by a stronger promoter may be performed using conventional genetic techniques (see general description above and Example 7 below for a specific protocol).

Insertion of an additional copy of the cel5Y gene in the Clostridium acetobutylicum genome may be performed by conventional genetic techniques (see general description above for the cip-cel operon and Example 8 below for insertion of an additional copy of the xynB gene).

Optional Overexpression of the xynB Gene

Alternatively or in combination with further overexpression of the cel9X gene and/or cel5Y gene, the genetically modified Clostridium acetobutylicum according to the invention may have been further genetically modified so that the xynB gene is overexpressed, its expression level in the genetically modified Clostridium acetobutylicum being higher than its expression level in a corresponding non-genetically modified Clostridium acetobutylicum.

The overexpression of the xynB gene is due to a genetic modification of the Clostridium acetobutylicum.

In order to overexpress the xynB gene, several types of genetic modifications may be made. In particular, the genetically modified Clostridium acetobutylicum according to the invention with an overexpressed xynB gene may be such that:
a) an additional copy of the xynB gene has been inserted in the Clostridium acetobutylicum genome under the control of a promoter stronger than the native promoter of the xynB gene,
b) the native promoter of the xynB gene has been genetically modified, preferably:
   b1) the native promoter of the xynB gene has been replaced by a stronger promoter, or
   b2) the native promoter of the xynB gene has been mutated.

The stronger promoter is preferably constitutive. Examples of constitutive promoters stronger than the native promoter of Clostridium acetobutylicum xynB gene include, without limitation, the promoter of Clostridium acetobutylicum thlA gene, ptb gene, crt gene and the gapC gene as presented in Table 2 above.

Replacement of the native promoter of the xynB gene by a stronger promoter may be performed using conventional genetic techniques (see general description above and Examples 1, 3 and 7 below for the cip-cel operon, the cel9X gene and the cel5Y gene).

Insertion of an additional copy of the xynB gene in the Clostridium acetobutylicum genome may be performed by conventional genetic techniques (see general description above for the cip-cel operon and Example 8 below for a specific protocol for insertion of an additional copy of the xynB gene).

As the xynB gene is located on Clostridium acetobutylicum megaplasmid, insertion of additional copy of the xynB gene in the Clostridium acetobutylicum genome under the control of a promoter stronger than the native promoter of the xynB gene is preferred.

Optional Further Overexpression of the cipA Gene, Resulting in an Increase of the Ratio of Produced CipA Scaffolding Protein to Produced Cellulases of the Cellulosome.

For optimum cellulolytic activity, all cellulases containing a dockerin domain should be attached to the scaffolding protein, via their dockerin-cohesin domains. In our case as C. acetobutylicum's scaffolding protein CipA contains five cohesin domains, this means that there should be one CipA protein for five dockerin containing cellulases.

When overexpressing the whole cip-cel operon, the inventors found that the ratio of scaffolding CipA protein to dockerin containing cellulases was not optimal for cellulolytic activity, and that further overexpressing the cipA gene encoding the scaffolding protein further permitted to improve the ratio and the cellulolytic activity.

Therefore, alternatively or in combination with further overexpression of one or more of the cel9X, cel5Y and xynB genes, the genetically modified Clostridium acetobutylicum according to the invention may have been further genetically modified so that the cipA gene is further overexpressed compared to other genes of the cip-cel operon. The modification preferably results in an increase of the ratio of produced CipA scaffolding protein to produced cellulases of the cellulosome. This in turn preferably results in a higher amount of cellulosome and a lower amount of free cellulases.

The cipA gene is already overexpressed due to the overexpression of the cip-cel operon, as cipA is a gene of this operon.

However, it may be further overexpressed compared to other genes of the cip-cel operon due to an additional genetic modification.

In order to further overexpress the cipA gene compared to other genes of the cip-cel operon, several types of genetic modifications may be made. In particular, the genetically modified Clostridium acetobutylicum according to the invention with a further overexpressed cipA gene compared to other genes of the cip-cel operon may be such that:
a) an additional copy of the cipA gene has been inserted in the Clostridium acetobutylicum genome under the control of a promoter stronger than the native promoter of the cipA gene,
b) The ribosome-binding site (abbreviated as “RBS”) of the cipA gene is changed to a stronger RBS. A stronger RBS sequence can be optimized for a given species and protein sequence using publicly available tools for example the Salis lab's RBS calculator (Reis A C, Salis H M. An Automated Model Test System for Systematic Development and Improvement of Gene Expression Models. ACS Synth Biol. 2020. 9 (11): 3145-3156.).
c)

The mRNA of the cip-cel operon is stabilized in such a manner to specifically increase the stability of the transcript encoding for cipA, as demonstrated in Xu et al. Cellulosome stoichiometry in Clostridium cellulolyticum is regulated by selective RNA processing and stabilization. Nat Commun. 2015. 6:6900 . . . . This paper shows that various stem loops or 3'UTR's are able to stabilize the mRNA transcript of R. cellulolyticum's cip-cel operon by protecting it from degradation by exonucleases. Additionally, they showed that this protecting effect is correlated with the free folding energy of the stem loop (ΔG). They determined the ΔG of the stem loops of multiple different species cip-cel operons including that of C. acetobutylicum. The stem loop between cipA and cel48A has a relatively high ΔG of only −20 compared to −33.8 for the stem loop between cipC and cel48F for R. cellulolyticum. Modifying C. acetobutylicum's cip-cel operon by changing the stem loop between cipA and cel48A for a stem loop with a lower ΔG for example the stem loop from between cipC and cel48F from R. cellulolyticum should result in increased stability of the mRNA transcript and thus increase the amount of CipA being produced.

In a preferred embodiment, in order to further overexpress the cipA gene compared to other genes of the cip-cel operon, an additional copy of the cipA gene has been inserted in the Clostridium acetobutylicum genome under the control of a promoter stronger than the native promoter of the cipA gene.

Insertion of an additional copy of the cipA gene in the Clostridium acetobutylicum genome may be performed by conventional genetic techniques (see general description above for the cip-cel operon and Example 10 below for a specific protocol for insertion of an additional copy of the cipA gene).

### Optional Inactivation of a Gene Encoding an Extracellular Protease

Alternatively or in combination with further overexpression of one or more of the cel9X, cel5Y and xynB genes, or in combination with further overexpression of one or more of the cel9X, cel5Y, xynB and cipA genes, in order to improve growth on lignocellulosic biomass, the genetically modified Clostridium acetobutylicum according to the invention may have been further genetically modified by inactivation of a gene encoding an extracellular protease. This is because extracellular protease may degrade the cellulosome, thus reducing the amount of cellulosome and limiting its ability to digest lignocellulosic biomass.

The extracellular protease is preferably selected from nrpE, CA_C0746, CA_C0625 and CA_C2695. More preferably, the extracellular protease is that encoded by gene nrpE, as this extracellular protease is the most expressed. In other preferred embodiments, several extracellular proteases can be inactivated in the same strain in order to further decrease the quantity of extracellular proteases, thus further decreasing degradation of the cellulosome. In this case, the inactivated extracellular proteases more preferably comprise the extracellular protease encoded by gene nrpE and one or more extracellular protease(s) encoded by the genes CA_C0746, CA_C0625 and CA_C2695.

Information regarding the genes encoding extracellular proteases that may be deleted in the genetically modified Clostridium acetobutylicum according to the invention in order to improve growth on lignocellulosic biomass is provided in Table 4 below.

TABLE 4
Clostridium acetobutylicum genes encoding extracellular
proteases that may be deleted in the genetically modified
Clostridium acetobutylicum according to the invention.

| | Name in | | Name in | |
|---|---|---|---|---|
| | NC_003030.1 | Position of | NC_015687.1 | |
| Conventional | (new/old | the gene in | (new/old | Position in |
| name | locus tag) | NC_003030.1 | locus tag) | NC_015687.1 |
| nrpE | CA_RS12895/ | 2625465 to | SMB_RS12435/ | 2551411 to |
| | CA_C2517 | 2627138 | SMB_G2468 | 2552712 |
| N/A | CA_RS04025/ | 866550 to | SMB_RS03965/ | 866530 to |
| | CA_C0746 | 868895 | SMB_G0762 | 868875 |
| N/A | CA_RS03395/ | 726150 to | SMB_RS03360/ | 726132 to |
| | CA_C0625 | 728027 | SMB_G0639 | 728009 |
| N/A | CA_RS13900/ | 2817677 to | SMB_RS13840/ | 2819302 to |
| | CA_C2695 | 2818174 | SMB_G2730 | 2819799 |

The inactivation of the extracellular protease is due to a genetic modification of the Clostridium acetobutylicum.

Inactivation of the extracellular protease may be obtained by any technique known in the art, including:
a) partial or complete deletion of the gene encoding the extracellular protease (in particular nrpE), and
b) insertion of inactivating mutations in the gene encoding the extracellular protease (in particular nrpE).
   Examples of inactivating mutations include insertion of an intron sequence or of a sequence encoding another protein in the sequence encoding the extracellular protease (in particular nrpE), or mutations at the active site of the extracellular protease (in particular nrpE) resulting in a decrease or suppression of protease activity.
   In the case of the extracellular protein encoded by the nrpE gene, the active site comprises amino acids 379E and 478H. Inactivation may thus be obtained by substitution of 379E and/or 478H by another amino acid, preferably another amino acid of a distinct type.

Partial or complete deletion of the gene encoding the extracellular protease (in particular nrpE) is the easiest mean to abolish protease activity and is thus preferred, and may be performed by conventional genetic techniques, including replacement techniques efficient in Clostridia based on homologous recombination as disclosed in WO2008040387 (with no sequence between the two homology arms) or ACE or CRISPR/Cas9 editing of Clostridium acetobutylicum genome (see Ehsaan et al. 2016 for ACE and Wilding-Steele et al. 2021 for CRISPR/Cas9 editing).

In case of partial deletion, at least 50% of the coding sequence is preferably removed, and more preferably at least 80%.

Insertion of inactivating mutations may be performed using conventional mutagenesis techniques, depending on the type of inactivating mutation.

Preferred Genetically Modified Clostridium acetobutylicum

The genetically modified Clostridium acetobutylicum according to the invention may combine several genetic modifications disclosed above as improving growth on lignocellulosic biomass.

In particular, preferred genetically modified Clostridium acetobutylicum strains according to the invention may be selected from:
a) A genetically modified Clostridium acetobutylicum in which the native promoter of the cip-cel operon has been replaced by the promoter of Clostridium acetobutylicum thlA gene.
   An example of such a strain is the SC1 Clostridium acetobutylicum strain obtained in Example 1 below.
b) A genetically modified Clostridium acetobutylicum in which the native promoter of the cip-cel operon has been replaced by the promoter of Clostridium acetobutylicum thlA gene and the native promoter of the cel9X gene has been replaced by the promoter of Clostridium acetobutylicum thlA gene.
   An example of such a strain is the SC4 Clostridium acetobutylicum strain obtained in Example 3 below.
C) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and the native promoter of the cel5Y gene has been replaced by the promoter of Clostridium acetobutylicum thlA gene,
   An example of such a strain is the SC6 Clostridium acetobutylicum strain obtained in Example 7 below.
d) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and an additional copy of the xynB gene has been inserted in the Clostridium acetobutylicum genome under the control of the promoter of Clostridium acetobutylicum thlA gene,
   An example of such a strain is the SC7 Clostridium acetobutylicum strain obtained in Example 8 below.
e) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and the nrpE gene has been partially or completely deleted.
   An example of such a strain is the SC5 Clostridium acetobutylicum strain obtained in Example 6 below.
f) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, and the nrpE gene has been partially or completely deleted.
   An example of such a strain is the SC8 Clostridium acetobutylicum strain obtained in Example 9 below.
g) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, an additional copy of the xynB gene has been inserted in the Clostridium acetobutylicum genome under the control of the promoter of Clostridium acetobutylicum thlA gene, and the nrpE gene has been partially or completely deleted.
   An example of such a strain is the SC9 Clostridium acetobutylicum strain obtained in Example 9 below.
h) A genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, an additional copy of the xynB gene has been inserted in the Clostridium acetobutylicum genome under the control of the promoter of Clostridium acetobutylicum thlA gene, an additional copy of the cipA gene has been inserted in the Clostridium acetobutylicum genome under the control of the promoter of Clostridium acetobutylicum thlA gene and the nrpE gene has been partially or completely deleted.
   An example of such a strain is the SC10 Clostridium acetobutylicum strain obtained in Example 10 below.

### Optional Further Genetic Engineering for Production of Specific Targeted Bulk Chemicals

The genetically modified Clostridium acetobutylicum according to the invention may be further genetically modified for an improved production of a targeted bulk chemical, based on genetic modifications known in the art for optimizing production of the specifically targeted chemical of interest, in particular by attenuating and/or deleting and/or replacing specific genes in order to favor a metabolic pathway for the production of the targeted bulk chemical.

Bulk chemicals susceptible to be produced by culturing a genetically modified Clostridium acetobutylicum according to the invention are known in the art and are preferably selected among the group consisting of ethanol, butanol, glycerol, 1,2-propanediol, acetone, isopropanol, isobutene, hydrogen, acetic acid and lactic acid and the like.

Non-limiting examples of additional modifications for optimizing production of ethanol, butanol and lactate are disclosed below, but the genetically modified Clostridium acetobutylicum according to the invention may also be further genetically modified to optimize production of other targeted chemicals of interest.

### Optimization for Ethanol Production

Suitable modifications for optimizing ethanol production are known in the art.

In particular, the genetically modified Clostridium acetobutylicum according to the invention may be further modified for optimized ethanol production by inactivation (including partial or complete deletion and insertion of inactivating mutations) of the lactate dehydrogenase (IdhA), thiolase (thlA) and hydrogenase (hydA) genes (see US20170240869A1, while this document does not mention inactivation of the IdhA gene, it should be noted that the IdhA gene has already been deleted in the strain sued for thlA and hydA inactivation).

Information regarding the thlA gene and the ptb gene have already been provided in Table 2 above.

Relevant information regarding and the hydA gene and the IdhA gene is provided in Table 5 below, which also includes information regarding the hbd gene and the buk gene, useful for optimizing butanol production.

TABLE 5
Clostridium acetobutylicum genes that may be further deleted
or overexpressed in the genetically modified Clostridium
acetobutylicum according to the invention in order to
optimize ethanol, butanol or lactate production.

| | Name in | | | Name in | | |
|---|---|---|---|---|---|---|
| | NC_003030.1 | Position of | Position of | NC_015687.1 | | Position of |
| Conventional | (new/old | the gene in | the promoter | (new/old | Position in | the promoter |
| name | locus tag) | NC_003030.1 | in NC_003030.1 | locus tag) | NC_015687.1 | in NC_015687.1 |
| hydA gene | CA_RS00180/ | 38101 to | 39850 to | SMB_RS00180/ | 38101 to | 39849 to |
| | CA_C0028 | 39849 | 40061 | SMB_G0028 | 39849 | 40024 |
| ldhA gene | CA_RS01485/ | 298777 to | 298596 to | SMB_RS01465/ | 298775 to | 298594 to |
| | CA_C0267 | 299718 | 298776 | SMB_G0272 | 299716 | 298774 |
| hbd gene | CA_RS13960/ | 831733 to | 2836452 to | SMB_RS13900/ | 2833358 to | 2838077 to |
| | CA_C2708 | 2832581 | 2836626 | SMB_G2743 | 2834206 | 2838250 |
| buk | CA_RS15800/ | 3225097 to | 3227098 to | SMB_RS08560/ | 1805558 to | 3228725 to |
| | CA_C3075 | 3226164 | 3227205 | SMB_G1685 | 1806628 | 3228912 |

The above modifications may be performed using conventional techniques (see US20170240869A1).

### Optimization for Butanol Production

Suitable modifications for optimizing butanol production are known in the art.

In particular, the genetically modified Clostridium acetobutylicum according to the invention may be further modified for optimized butanol production by inactivation (including partial or complete deletion and insertion of inactivating mutations) of the ptb-buk operon, replacement of the Clostridium acetobutylicum thlA gene by Escherichia coli atoB gene, replacement of the Clostridium acetobutylicum hbd gene by Clostridium kluyveri hbd1 gene, and overexpression of CA_C0764 gene, its expression level being higher than its expression level in a corresponding non-genetically modified Clostridium acetobutylicum (see Nguyen et al. 2018; Foulquier et al, 2022).

The sequence of Escherichia coli atoB gene may be found in NC_000913.3 (position 2326109 to 2327293).

The sequence of Clostridium kluyveri hbd1 gene may be found in CP000673.1 (position 437268 to 438116).

The sequence of Clostridium acetobutylicum CA_C0764 gene corresponds to positions 886021 to 887256 of NC_003030.1.

These modifications may be performed using conventional techniques (see e.g. Nguyen et al. 2018; Foulquier et al, 2022).

### Optimization for Lactate Production

Suitable modifications for optimizing lactate production are known in the art.

In particular, the genetically modified Clostridium acetobutylicum according to the invention may be further modified for optimized lactate production inactivation (including partial or complete deletion and insertion of inactivating mutations) of the thiolase (thlA) and hydrogenase (hydA) genes followed by curing of the megaplasmid (see US20170240869A1, please note that in this document, IdhA gene was first restored because the starting strain was deleted for ldhA). If the genetically modified Clostridium acetobutylicum according to the invention is inactivated for IdhA gene, then this gene should also be restored.

In the context of the present invention, the term “curing” used in conjunction with a megaplasmid, means the elimination of the said megaplasmid from the cell of the microorganism containing by appropriate means that are, for example, targeting an appropriate sequence on the megaplasmid using CrispR-Cas9 (see also those disclosed in US20170240869A1).

Method for the Production of a Targeted Bulk Chemical from Ligoncellulosic Biomass

The present invention also relates to a method for the production of a targeted bulk chemical from lignocellulosic biomass, comprising culturing a genetically modified Clostridium acetobutylicum according to the invention on an appropriate culture medium comprising lignocellulosic biomass as main source of carbon, and recovering the targeted bulk chemical from the culture medium.

Known appropriate culture media for cultivation of Clostridium acetobutylicum include, without limitation, the Clostridial Growth Medium (CGM) medium (Dusséaux et al. 2013), 2XYT (Oultram et al. 1988), Minimal synthetic (MS, Soni et al. 1987), and Clostridial Basal Medium (CBM, see O′BRIEN et al. 1971).

In the above method, lignocellulosic biomass has preferably been pre-treated by one of many pre-established methods, including steam explosion, ammonia fiber expansion, alkali extraction (e.g. alkali extracted deshelled corn cobs, also referred to as “AECC”), organosolv treatment, or sulfite treatment (Zhao et al. 2022).

Bulk chemicals susceptible to be produced by culturing a genetically modified Clostridium acetobutylicum according to the invention are known in the art and are preferably selected among the group consisting of ethanol, butanol, glycerol, 1,2-propanediol, acetone, isopropanol, isobutene, hydrogen, acetic acid and lactic acid and the like.

Depending on the specifically targeted bulk chemical(s), the skilled person will know which genetically modified Clostridium acetobutylicum according to the invention is the more appropriate.

The following examples merely intend to illustrate the present invention.

### EXAMPLES

### Example 1: Clostridium acetobutylicum Native Cel48A Protein is Active

1: Cel48A Production in E. coli Results in a Misfolded Protein

EP2436698A1 previously stated that Cel48A was not functional. We demonstrated that Cel48A was indeed functional. In EP2436698A1, the sequence cloning for Cel48A was purified from E. coli and was shown to have very low activities on CMC and PASC with no activity on crystalline cellulose.

We later demonstrated that in fact Cel48A purified in this manner was not correctly folded possibly explaining the lack of activity. Specifically, although when Cel48A was initially purified it appeared soluble, however, after ultracentrifugation (at 100,000 RPM) 100% of Cel48A was found in the insoluble fraction. This was not the case for Cel48F from R. cellulolyticum which was purified at the same time as a control.

2: Cel48A Expression in Clostridium acetobutylicum

The activity of Cel48A was determined by purifying Cel48A directly from C. acetobutylicum. Firstly, the native promoter of the cip-cel operon was replaced with the constitutive thiolase (thlA or thl) promoter (pthl), creating strain SC1. To achieve this the strategy described by (Wilding-Steele et al. 2021) was used, CAS2ΔldhA was used as the base strain and plasmid pGRNA_pthl-cip-cel to perform the genetic modification. This plasmid was constructed by Genewiz using cassette pGRNAJ23119Δupp as a template

(Wilding-Steele et al. 2021), the cassette was digested with SacI/EcoRI and the synthetic sequence synthesized by Genewiz was inserted. The synthetic sequence of plasmid pGRNA_pthl-cip-cel is defined as SEQ ID NO:1.

Secondly, second copy of orfXp and a terminator were inserted between cel48A and cel5B, creating strain SC2, this resulted in a truncated transcript resulting in only cipA, cel48A and orfxp being expressed. This was achieved using SC1 as the base strain with plasmid pGRNA_cel48A_T1T2. This plasmid was constructed by Genewiz using cassette pGRNAJ23119Δupp as a template, the cassette was digested with SacI/EcoRI and the synthetic sequence synthesized by Genewiz was inserted. The synthetic sequence of plasmid pGRNA_cel48A_T1T2 is defined as SEQ ID NO:2.

Lastly a mutation was introduced in Cel48A which replaced the glutamic acid at position 66 for glutamine creating strain SC3. This is the equivalent of Cel48F_E55Q which has been shown to result in an inactive enzyme as E55 is the proton donor. This was achieved using SC2 as the base strain and using plasmid pGRNA_cel48A_E61Q. This plasmid was constructed by Genewiz using cassette pGRNAJ23119Δupp as a template, the cassette was digested with SacI/EcoRI and the synthetic sequence synthesized by Genewiz was inserted. The synthetic sequence of plasmid pGRNA_Cel48A_E61Q is defined as SEQ ID NO: 3.

### 3: Catalytic Properties of the CipA:Cel48A and CipA:Cel48A_E61Q Complex

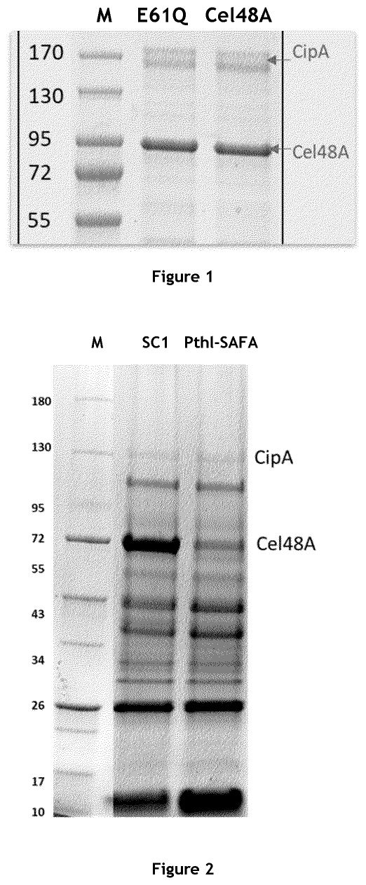

The CipA:Cel48A complex was purified from strains SC2 and SC3 by growing in MS medium (Soni et al. 1987), the CipA:Cel48A complex was then purified using cellulose affinity chromatography. The purity of this complex was validated by SDS-PAGE and proteomic analysis (FIG. 1).

The specific activities of the CipA:Cel48A complexes were studied using Phosphoric Acid Swollen Cellulose (PASC) and Avicel (crystalline cellulose) as substrates. The degradation activities were followed for at least two hours. The results are summarized in Table 6 and compared with the activities of homologous Cel48F which is known to act efficiently on crystalline cellulose (Reverbel-Leroy et al. 1997). Activity assays showed that the CipA:Cel48A complex was active on PASC and Avicel while the CipA:Cel48A_E61Q complex showed very low activity (as expected). This shows that Cel48A is secreted in an active form by C. acetobutylicum.

TABLE 6
Activity of the CipA:Cel48A complex and CipA:Cel48A_E61Q
complex on various substrates and comparison with activities
of previously characterized GH48 cellulase from
R. cellulolyticum.

| Activity | | | |
|---|---|---|---|
| (mU/mg) | Cel48A | Cel48A_E61Q | Cel48F |
| PASC | 25.7 | 2.7 | 130 |
| Avicel | 4.8 | 1.3 | 3.2 |

### Example 2: Cel48A is Expressed at Significantly Higher Levels Compared to Cel48A_SAFA

The C. acetobutylicum SAFA strain disclosed in EP2436698A1, in which cel48A has been replaced with cel48SAFA was genetically modified by replacing the promoter of the cip-cel operon by the thiolase (thlA) promoter, creating strain C. acetobutylicum Pthl-SAFA. The expression level of the cellulosomal components in this strain and in strain SC1 (identical excepted for the fact that the native celA48 gene is present instead of the hybrid cel48SAFA gene) were compared and Cel48SAFA was shown to be produced at significantly lower levels compared to native Cel48A (FIG. 2).

### Example 3: Expression of Cel9X in Clostridium acetobutylicum

The native promoter of cel9X was changed to the thiolase promoter, strain SC1 was used as the base strain and the modification was performed using plasmid pGRNA_pthl-cel9X, creating strain SC4. This plasmid was constructed by Genewiz using cassette pGRNAJ23119Δupp as a template (Wilding-Steele et al. 2021), the cassette was digested with SacI/EcoRI and the synthetic sequence synthesized by Genewiz was inserted. The synthetic sequence of plasmid pGRNA_pthl-cel9X is defined as SEQ ID NO:4.

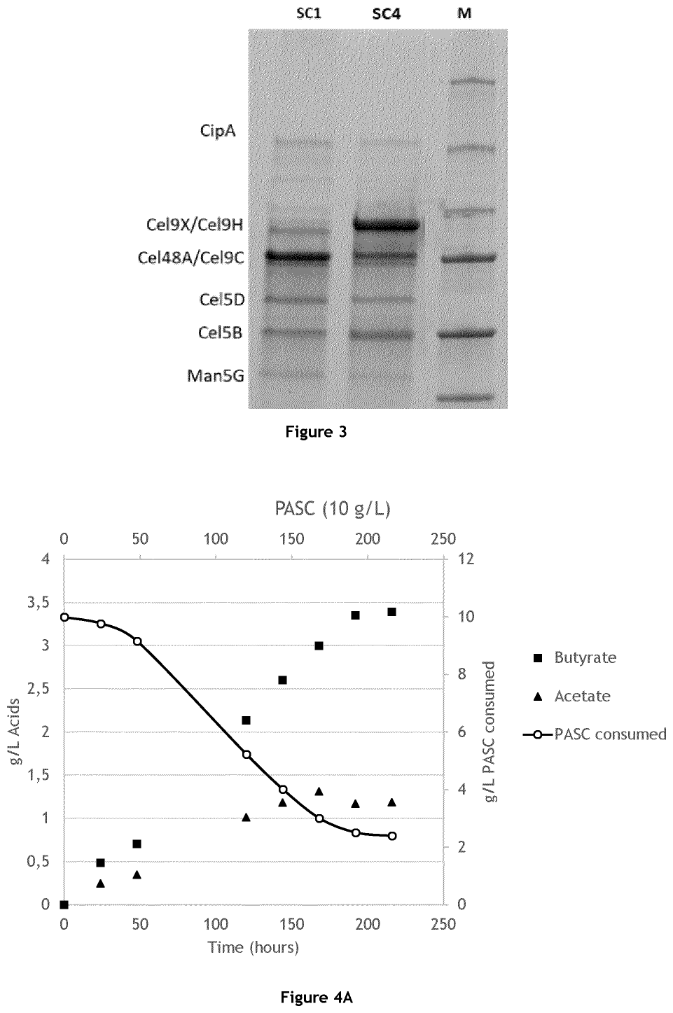

Analysis of the supernatant showed that Cel9X was produced at high levels, in addition to proteins present in the cip-cel operon (FIG. 3).

### Example 4: Catalytic Activities of the Cellulosomes Produced from Recombinant C. acetobutylicum

Cellulosomes from SC1, SC4 and R. cellulolyticum were purified using cellulose affinity chromatography.

Activity assays showed that cellulosomes from SC1, SC4 and R. cellulolyticum showed similar activity on PASC and Avicel (Table 7).

TABLE 7
Activity of the cellulosome purified from stains SC1 and SC4
on various substrates and comparison with the activities of
previously characterized cellulosome from R. cellulolyticum.

| Activity | | | R. |
|---|---|---|---|
| (mU/mg) | SC1 | SC4 | cellulolyticum |
| PASC | 281 | 403 | 368 |
| Avicel | 9.4 | 19.3 | 39 |

### Example 5: The Recombinant Strain is Able to Grow on Lignocellulosic Biomass

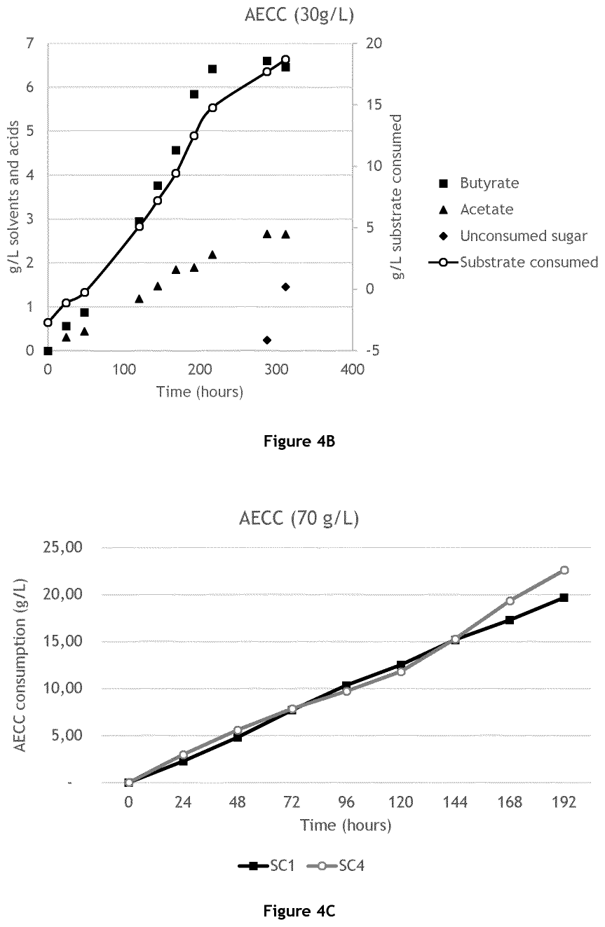

Strain SC4 was assessed for its ability to grow on lignocellulosic biomass. Small scale flask fermentations were performed in which the cells were grown in CGM medium containing 5 g/L PASC or 30 g/L AECC. SC4 was able to grow on PASC and AECC (FIGS. 4A and 4B). Finally, strain SC4 was shown to grown faster on AECC compared to strain SC1, especially at the end of the culture presumably as the addition of Cel9X resulted in the strain able to degrade the more recalcitrant cellulose (FIG. 4C).

### Example 6: Deletion of an Extra-Cellular Protease Results in Improved Growth on Lignocellulosic Biomass

nrpE is a gene encoding an extracellular protease which may degrade the cellulosomal components. The gene nrpE was deleted using strain SC4 as the base strain. The modification was performed using plasmid pGRNA_AnrpE, creating strain SC5. This plasmid was constructed by Genewiz using cassette pGRNAJ23119Δupp as a template (Wilding-Steele et al. 2021), the cassette was digested with SacI/EcoRI and the synthetic sequence synthesized by Genewiz was inserted. The synthetic sequence of plasmid pGRNA_AnrpE is defined as SEQ ID NO:5.

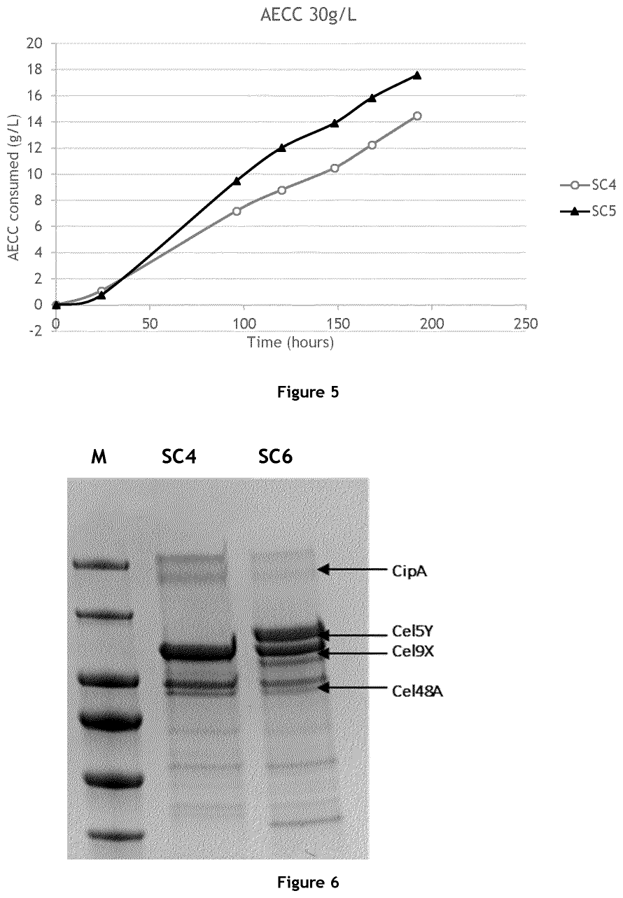

Strain SC4 and SC5 were grown on AECC and strain SC5 was shown to grow significantly faster (FIG. 5). Additionally, a higher amount of cellulosome was produced in strain SC5 compared to strain SC4 (data not shown).

### Example 7: Expression of Cel5Y

Cel5Y is another cellulosomal cellulase, which we found to be able to bind the cellulosome to the cell wall. On this basis, it was hypothesized that its overexpression might further improve growth on lignocellulosic biomass.

cel5Y was overexpressed by changing its native promoter to the strong thiolase promoter (pthl). This was performed using strain SC4 as the base strain. The modification was performed using plasmid pGRNA_pthl_cel5Y, creating strain SC6. This plasmid was constructed by Genewiz using cassette pGRNAJ23119Δupp as a template (Wilding-Steele et al. 2021), the cassette was digested with SacI/EcoRI and the synthetic sequence synthesized by Genewiz was inserted. The synthetic sequence of plasmid pGRNA_pthl_Cel5Y is defined as SEQ ID NO:6.

The cellulosomes were purified from SC4 and SC6 (FIG. 6) and the cellulosomes purified from SC6 were shown to have higher levels of activity on PASC and Avicel (Table 8).

TABLE 8
Activity of the cellulosome purified from
stains SC4 and SC6 on various substrates.

| Activity (mU/mg) | SC4 | SC6 |
|---|---|---|
| PASC | 403 | 1333.3 |
| Avicel | 19.3 | 25.6 |

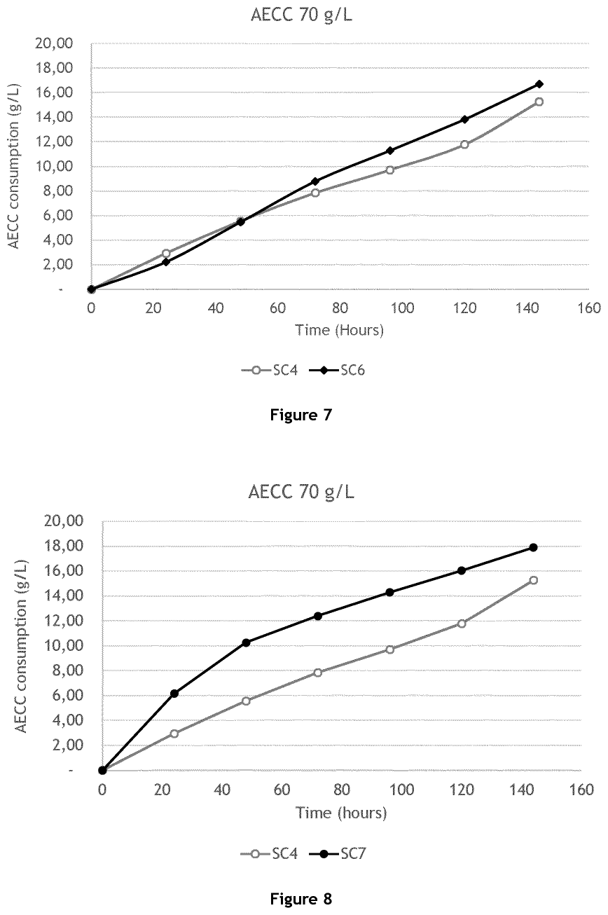

Additionally, strains SC4 and SC6 were grown on AECC and strain SC6 was shown to grow faster (FIG. 7).

### Example 8: Expression of xynB Results in Improved Growth on Lignocellulosic Biomass

As the objective was to grow on pre-treated lignocellulose, which contains both cellulose and hemi-cellulose, it was hypothesized that co-expression of xylanases would increase the growth rate on pre-treated lignocellulose.

One of C. acetobutylicum's native xylanases, XynB was overproduced by cloning an extra copy of the xynB gene downstream of the thiolase gene. This was performed using strain SC4 as the base strain. The modification was performed using plasmid pMTL_JH16-xynB, creating strain SC7. This plasmid was constructed using plasmid pMTL_JH16 (GenBank accession number HQ875757.1) as the template, a PCR was performed using oligos 1 and 2 (oligo 1=XynB-not1: agcggccgcAATTAATTATTAAATAAA (SEQ ID NO:7); oligo 2=XynB-Nhe1: gctagcaaagCTAATGTGATGCT (SEQ ID NO:8)) and using C. acetobutylicum's gDNA as a template. The cassette and PCR product were digested with Not1/Nhe1 and ligated together.

SC4 and SC7 were grown on AECC and strain SC7 was shown to grow significantly faster (FIG. 8).

### Example 9: Construction and Characterization of Strains SC8 and SC9 Combining Overexpression of the Cip-Cel Operon, Cel9X, Cel5Y (and Optionally xynB) and Deletion Of Extra-Cellular Protease NrpE

Strain SC8 was constructed using strain SC6 as the base strain. The modification was performed using plasmid pGRNA_AnrpE, (see Example 6) creating strain SC8.

Strain SC9 was constructed using strain SC8 as the base strain. The modification was performed using plasmid pMTL_JH16-xynB, (see Example 8) creating strain SC9.

### Example 10: Addition of a 2^{nd} Copy of cipA the Scaffoldin Gene Results in Improved Growth on Lignocellulosic Biomass

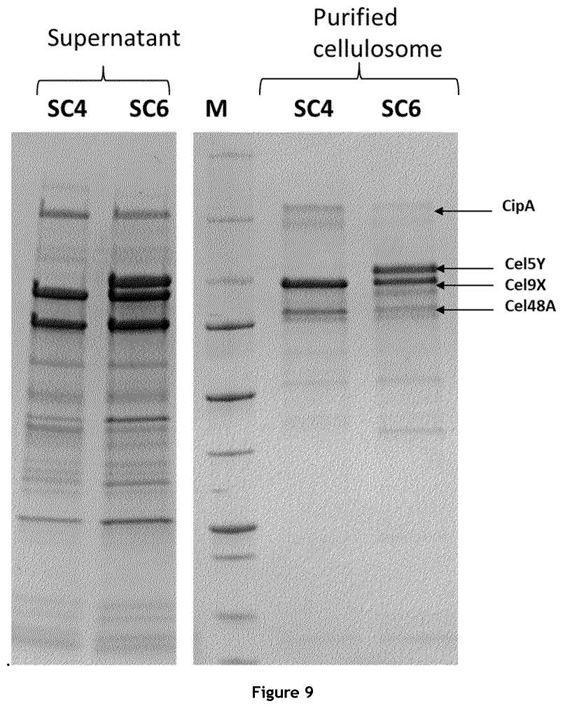

Preliminary analysis using SDS-PAGE showed that the cellulosome was probably saturated, this means that there was an excess of dockerin containing proteins compared to cohesin domains, meaning that not all cellulases could bind to the scaffoldin protein CipA. This is shown as there is an obvious decrease in the intensity of the band corresponding to Cel48A in the purified cellulosome fraction compared to the supernatant. It should be noted that high levels of Cel9X and Cel5Y are probably not bound to the cellulosome but are co-purified with the cellulosome as they contain a cellulose binding domain while Cel48A does not. (FIG. 9). This could result in a decrease in the activity and stability of the cellulases as binding of the cellulases to a scaffoldin protein has previously shown to increase their activity and stability.

In order to increase the amount of CipA produced a second copy of cipA was introduced downstream of the thiolase gene, this was in an operon with xynB. The modification was performed in strain SC8 (which was constructed by deleting nrpE in strain SC6) using plasmid pMTL_JH16-cipA-xynB, creating strain SC10. SC9 was also constructed as a control using plasmid pMTL_JH16-xynB. pMTL_JH16-cipA-xynB plasmid was constructed using plasmid pMTL_JH16-xynB as the template, a PCR was performed using forward primer cipa-not1_fw: GCAGATGGCGGCCGCggcccagaatttaaaaggagg (SEQ ID NO:11) and reverse primer cipa-not1-rev: caacaaatggaaaaataactgttgaataaGCGGCCGCatttc (SEQ ID NO: 12) and using C. acetobutylicum's gDNA as a template. The cassette and PCR product were digested with Not1 and ligated together.

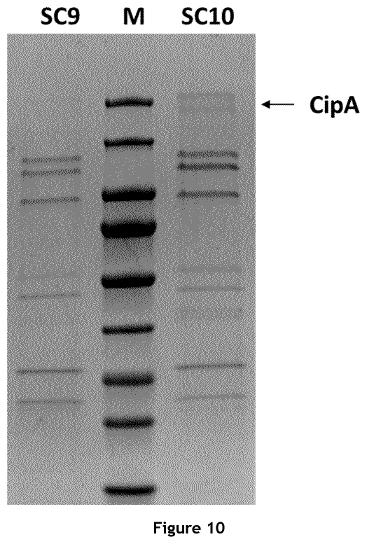

StrainsSC9 and SC10 were grown in triplicate in MS-cellobiose to an OD600 of 1.5. The supernatant was then collected by centrifugation and was subsequently concentrated and dialyzed in Sodium Acetate buffer (20 mM Sodium Acetate, 10 mM CaCl2), 100 mM NaCl, pH 5.5) using ultrafiltration (Vivaspin Turbo 15 10,000 MWCO). Protein concentration was determined using Bradford and the concentration of protein was normalized to 0.1 mg/ml. The supernatant was then analyzed by SDS-PAGE and quantitative proteomic analysis. SDS-PAGE showed that the band at 180 KdA probably corresponding to CipA showed increased intensity for strain SC10 compared to SC9 (FIG. 10). Quantitative proteomic analysis was performed using a TripleTOF 6600 mass spectrometer (Sciex). Sequential window acquisition of all theoretical spectra (SWATH) and additional data processing was performed using DIA-NN. In order to be able to compare the number of individual proteins produced, the molar amount of each protein was determined, and then the molar percentage was calculated by dividing the molar amount of each individual protein by the molar amount of all supernatant proteins. The proteomic analysis consequently showed an almost 3-fold increase in the amount of CipA in strain SC10 compared to SC9 (Table 9). Analysis of the data showed that the cohesin/dockerin ratio was 0.54 for SC9 and 1.54 for strain SC10 (this ratio was calculated by determining the number of cohesin domains (moles of CipA multiplied by 5) divided by the total number of dockerin-containing proteins). This confirms that for strain SC10 there is sufficient production of CipA, which is not the case for strain SC9.

TABLE 9
Molar percent of cellulosomal proteins found
in the supernatant of strains SC9 and SC10

| Cellulosomal protein | SC9 | SC10 |
|---|---|---|
| CipA | 0.79 | 2.36 |
| Cel48A | 2.81 | 2.87 |
| Cel9X | 1.40 | 1.84 |
| Cel5Y | 1.59 | 1.46 |
| Cel5B | 0.83 | 0.70 |
| Cel9C | 0.23 | 0.18 |
| orfXp | 0.00 | 0.00 |
| Cel44D | 0.08 | 0.09 |
| Cel9E | 0.02 | 0.06 |
| Cel9F | 0.04 | 0.11 |
| man5G | 0.21 | 0.27 |
| cel74h | 0.11 | 0.08 |
| Total cellulosome | 8.12 | 10.01 |
| Total dockerin-containing proteins | 7.33 | 7.66 |
| Total cohesins | 3.94 | 11.78 |
| Dockerin/Cohesin ratio | 0.54 | 1.54 |

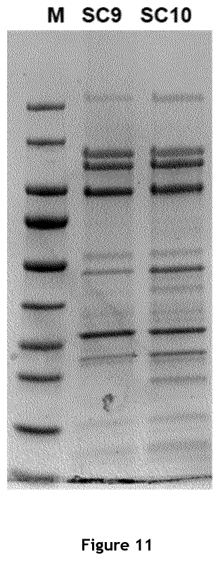

The cellulosomes were purified from SC9 and SC10 (FIG. 11) and the cellulosomes purified from SC10 were shown to have higher levels of activity on Avicel than the cellulosomes purified from SC9 (Table 10), showing that adding a 2nd copy of the scaffoldin gene cipA results in improved activity on crystalline cellulose.

TABLE 10
Activity of the cellulosome purified from
strains SC9 and SC10 on various substrates.

| Activity (mU/mg) | SC9 | SC10 |
|---|---|---|
| PASC | 1423 | 1249 |
| Avicel | 27 | 30.5 |

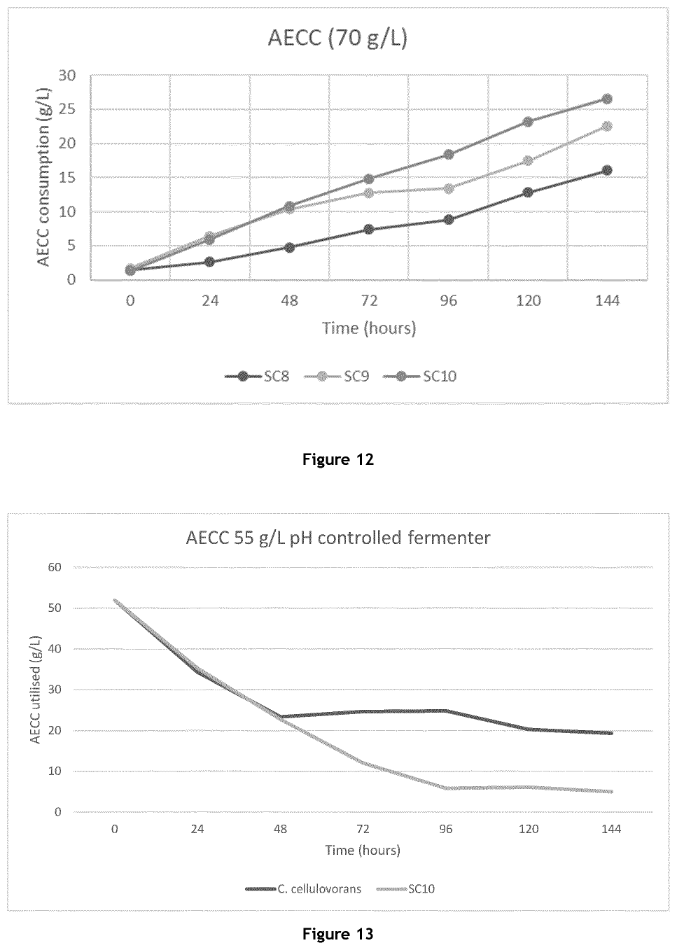

Additionally, strains SC8, SC9 and SC10 were grown on AECC and strain SC10 was shown to grow faster (FIG. 12, showing that adding a 2nd copy of the scaffoldin gene cipA results in improved growth on lignocellulosic biomass).

The growth of C. acetobutylicum strain SC10 on AECC (in pH-controlled fermenters with 55 g/L AECC) was also compared to the growth of C. cellulovorans in the same conditions.

FIG. 13 shows that C. acetobutylicum strain SC10 is much more efficient than C. cellulovorans to utilize AECC.

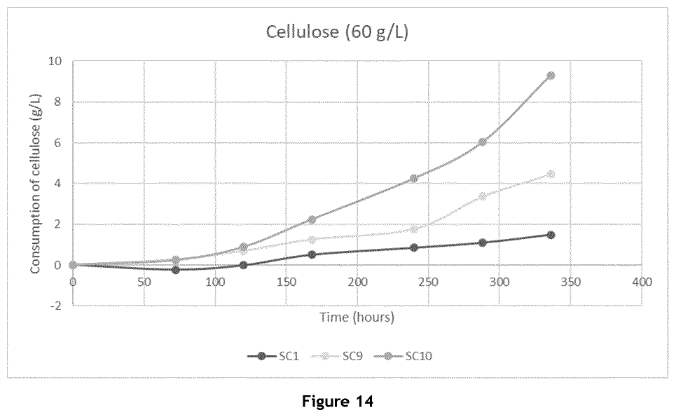

Finally, C. acetobutylicum strain SC10 was shown to be able to grow on crystalline cellulose, which is not the case of C. acetobutylicum strains SC1 and SC9 which are only able to grow very poorly (FIG. 14).

### List of Strains Used in Examples

Strains used in Examples are listed in Table 11 below.

TABLE 11
Description of C. acetobutylicum strains used in Examples.

| Strain name | Modifications |
|---|---|
| CAS2delldhA | None, this strain is the initial C. acetobutylicum strain |
| | used for constructing strains SC1 to SC10. |
| SC1 | Pthl-Cipcel |
| SC2 | Pthl-cipcel_T1T2 |
| SC3 | Pthl-cipcel_Cel48A_E61Q_T1T2 |
| Pthl-SAFA | Pthl-cipcel(SAFA) |
| SC4 | Pthl_cipcel, Pthl-cel9X |
| SC5 | Pthl_cipcel, Pthl-cel9X, ΔNrpE |
| SC6 | Pthl_cipcel, Pthl-cel9X, Pthl-Cel5Y |
| SC7 | Pthl_cipcel, Pthl-cel9X, +XynB |
| SC8 | Pthl_cipcel, Pthl-cel9X , Pthl-Cel5Y, ΔNrpE |
| SC9 | Pthl_cipcel, Pthl-cel9X , Pthl-Cel5Y, |
| | ΔNrpE, +xynB |
| SC10 | Pthl_cipcel, Pthl-cel9X , Pthl-Cel5Y, |
| | ΔNrpE, +cipA-xynB |

Pthl-cipcel: replacement of the native cip-cel operon promoter by the thiolase promoter (pthl) in the native cip-cel operon. T1T2: insertion of a terminator and a second copy of orfxp between cel48A and cel5B. cel48A_E61Q: replacement of glutamic acid in position 66 of Cel48A by a glutamine. Pthl-cipcel (SAFA): replacement of the native cip-cel operon promoter by the thiolase promoter (pthl) in the SAFA cip-cel operon (comprising a hybrid cel48SAFA gene instead of the native cel48A gene). Pthl-cel9: replacement of the native cel9X gene promoter by the thiolase promoter (pthl). ΔNrpE: deletion of the NrpE gene. Pthl-cel5Y: replacement of the native cel5Y gene promoter by the thiolase promoter (pthl). +xynB: cloning of an extra copy of the xynB gene downstream of the thiolase gene. +cipA-xynB: cloning of an extra copy of the cipA and xynB genes downstream of the thiolase gene.

### List of Plasmids Used in Examples

Plasmids used in Examples and their sequences are listed below.

SEQ ID NO: 1-pGRNA_Pthlcipcel:
GAGCTCTTGACAGCTAGCTCAGTCCTAGGTATAATACTAGTACCATTAATTTAATAAAAGGGTTTT
AGAGCTAGAAATAGCAAGTTAAAATAAGGCTAGTCCGTTATCAACTTGAAAAAGTGGCACCGAGT
CGGTGCTTTTTTTGTCGACTAAATGCAGCAATTGAAGCTGCTAGAGCGGGAGAAGCAGGAAAGG
GATTTTCTGTGGTGGCTGAAGAAATTGGAAAGCTTGCAGAGGAATCAACATCAGCAACAAAAGAG
GTTAAAAAACTTCTTGAAGATATAAAAAGTAAAAATAGTGTAGTATTTAAATCAATAGATGTTTCT
CTAAAATTATCAGAGGAACAAACTGAATCAGTTAAAGAGACTAAAGAAATATTTAATAAGATATTA
GAGTCTTTAAATAGTTTAGTAGGAGAAATAGAAAATATAAGGGGCTCTATAAATGACACTTATCA
GAGTAAGAACTTTATATTTGGTAAGTTAGAAAGCATATCAGCAGCATCAGAACAATCAGCATCTA
GTTCTGAGGAAGTATCTGCAACTACAGAAGAAGTATCAGCTAGTATGAGTGAATTTAATAAGATG
TCTCAAAAATTAGAACAAGTTGTAGAGGAACTTGAGAGAGAAATACAAAACTTTACGCTGTAAGC
TGGCAGAAGTCACTGAAGCGGCCGACTTTTTAACAAAATATATTGATAAAAATAATAATAGTGGG
TATAATTAAGTIGTTAGAGAAAACGTATAAATTAGGGATAAACTATGGAACTTATGAAATAGATTG
AAATGGTTTATCTGTTACCCCGTAGGGCCCAGAATTTAAAAGGAGGGATTAAAATGCGTAAAAAG
TCTTTAGCATTTTTGTTAGCACTAACAATGTTGGTGACATTATTAGGGGCTCAGCTTACAGCTTTT
GCAGCAGGTACTGGCGTCGTTCAAATACAATTTGCTGATACAAATACTAGTACAACCATGAATACT
ATTGCTCCTAAATTTAAAATCACAAATAATACTGGAGCACCTTTAGATTTAACAACTTTAAAATTAA
GATACTATTTTACAGCTGATGGTACTCAGGATGAAAATTTTTGGTGCGACCATGCTGGTATGCTT
AATGGTTATAACTACCAAACAATTACAAGTAATGTAGTGGGTACTTTTGTAGCTATGGATAATGCA
ACAGCTACTGCTGATCATTATCTTGAGATAAGCTTCTCAAATGGAGCAGGACAACTTGATGCAGG
TTCTTCACTTGAAGTTCAATGCAGAGTTGCAAAGAATGACTGGAGTAATTATGATCAATCAAACG
ATTATTCATTTACTTCTAATGCAAGTGATTTTACTGACTGGGATAAGATAACAGGTTATGTTAATG
GAGATCTTGTATTCGGAAATCCACCAGTAGTAGACCCAGTTATTACTCCAACTACTGCGAATTC
-pGRNA_cel48A_T1T2
SEQ ID NO: 2
gagctcccgcggtgcggccgcgggccctactttttaacaaaatatattgataaaaataataatagtgggtataattaagttgt
tACTAAATGTAAAATGTTAGCgttttagagctagaaatagcaagttaaaataaggctagtccgttatcaacttgaaaa
agtggcaccgagtcggtgcttttttgtcgacgcggccgctctagaactagtgGATCcttgccgaaaaagggatctgacagta
aatccatcaggtttcattgttgcagatgaaaatgataaggatgtagatcatgcatcaacagatggaaaaataacaatcacag
gttcagcaccagttgttgtaaattcgtctgtaaatacttctagtgtaacttatgatccagcagcaccacaggatcaagctgtc
agtgttacacttaatggcaatacaatcacagatgtaaaggacgcaaacgctagtgttcttaaggctggaagtgattacacag
taacaagtgatggaattacactaagccaaagctatcttgctactctagcagcaggaacttacacatatacagttgattttagt
gcaggaaatgcaggtacatttactgttgttgttaagggaaaagcagtagtaaataaaactactttagcagtaggagctgcat
caggaaaagcaggagatactgttaaggtgcctgtaactataagtaaagtaacaacaccagtaggtttaatatgcatggaaat
agattatgatgcaagtaagtttactgttaaggatgtacttcctaatacagatcttgtaaaagatactgataactacagctttat
tgctaatacgacatcagcaggaaaaatcagtattacatttacagatccaacacttgagaaattcccaataagtgcagatgga
gttatagcaaatatagattttgttgtaaattcaggtgcagcaactggtgatagcgatttaacagtaaattcatcaggtttcatt
gttgcagatgaaagtgatacagatatagatcatgtatcaacaaatggaaaaataactgttgaataatcaatgacataatact
gccgccattataggatagcggcagtatactataaaattttaattaattatttttaaaggagatagaaaaatatgttaaagata
agtaagaattttaaaaaaataatggctgtagctcttacatctacagttatatttggttctctatctggattattaactaataaa
gttgcagctgctacaactacagattcatccttaaaagtagataatgcgtatactcaaagatttgaaacaatgtacaatAGAa
tgcacgatgctaataatggatatttcagtaaagatggtgttccatatcactcagttgaaacctttatggttgaggcacctgatt
atggtcatgaaactacaagtgaagctttcagttattatatgtggcttgaagcaatgcagggaaaaatcacaggaaactttagt
ggagtgaataccgcatgggatacagctgaaaagtatatgattccatcacaccaagatcaaccaggtatggatagatataac
gctagtagtccagcaacatattcaccagaatgggaagatccaagtaagtatccatctagaatggatcaaggagcagcaaaa
ggacaagatccaataagtgatgagcttaaatcagcatatggaacttcagatatgtatggaatgcattggttaatggatgttga
caactggtatggctttggaaatcatgaagatggaacatctaaaaatgtttatataaacacttatcaaagaggagaacaggaa
tctgtttttgaaacagtacctcaaccatgttgggatgctttcaaatatggtggaaaaaatggatatcttgacttattcacagga
gataatagctatgcaaaacaagctaaatacacagatgcaccagatgctgatgctcgtgcaatacaggcaacttatgaagca
gcacaagcagctaaagaggatggagtagatttaagctcaatcgtaggtaaagcttctaaaatgggagattacttaagatatg
ctatgtttgataaatattttagaaaaataggaaattcaactcaagcaggaaatggtaaagattcaatgcattatttactttctt
ggtattatgcttggggtggatctcaaaataatgattggtcttggaaaataggctgcagtcacagtcattttggatatcaaaatc
ctttaactgcatgggtactttcaactgatagtcaattcaaacctaaatcagcaactggtgcaactgactgggcaaagagttta
acaactcaggtagatttctatcaatggttacaatcttcagagggagctatagcaggtggtgctagtaactcaaatcatggtcg
ttatgaagcatggccagaaggtacagctacatttgatggaatgggatatcaagaagaaccagtttaccatgatccaggtagt
aacacatggtttggaatgcagtcatggtcaatgcagcgtatggctcaatactactatcaatcaaaagacccaaaagcaaaa
gctttacttgacaaatgggttaaatggattaaatctgtagttaaagtaaatccaaatggtgcaggaacatttgaagtaccatc
aaaattaagttggactggacaaccagatacatggacaggatcttatacaggaaatcctaacttacatgtaaatgttgattcat
atactactgatataggtacaagctcatcaactgcagatgcacttgcatactatgcagcagctacaggagataaggattcaca
agcactttcaaaaactatacttgatgatatctggaaaaactatcaggatgcaaagggtgtatcagcaccagaacaaatggac
tatagccgtgtattcaatcaagaagtttatatcccacaaggttggacaggaacaatgcctaatggagatgtaattaaatcag
gaaataaattcatagacatacgttcacaatataagaatgatcctgattatgctagagttaaatctgatgttgaagcaggaaa
gtcaagtacatttaactatcatcgtttctgggcagaaagtgagtatgctatagcaaatgcaaattatggaactttattcgctaa
tacagctacaccaggagatgtaaatggagacggcgtagttaatggaagagatcttatggagcttagacagtatttagcaggt
aagttagatgctagtaaaataaatttagctgcagcagatgttaataatgatggtgtagttaatggaagagatattatggaact
tactaaattaattgcaaaatagatcttataagaaaataacggaaggaagagtattcaatgaagaaaaaaggaataattaca
gcagtaatattgagtttactagttattggtacggtaggttgtaaatcaaacgacacaaagagtactgtatccgctaatcttgta
agcagtgaaaagtctaatttaaagataagcagtagtagcggaaaaacaggaagtaatgtggaggttaaagttaaggcaagt
aatgtagctaaaaagaatgttatttgctgcgattttaaaataaaatatgatgctagtaaattagacgtttctggtatttcacca
ggagaaattctaaaagatcctaaagataaccttgagtacaatgtggatagcaaaaatggtgttattacaattttatattcgta
ctcagataaaaatataggaaaagaattgatatcaaaaaatggggattttgtaactttaaacttgaaaataaaagatgatgct
aaaaagggaaaaactcaaataagctttaatggcaatcctgaattctatgataagaatcaaaaaggtgtgtcagttactacta
ataaaggtgaaatagaaattaaataggCcaggcatcaaataaaacgaaaggctcagtcgaaagactgggcctttcgtttta
tctgttgtttgtcggtgaacgctctcctgagtaggacaaatccgccgggagcggatttgaacgttgcgaagcaacggcccgga
gggtggcgggcaggacgcccgccataaactgccaggcatcaaattaagcagaaggccatcctgacggatggcctttttgcgt
ttcttagtagaatcttaatttatgggagaagtgtttgatatgaaaaaaaaagttttgcttaagtgctatttttgcatcgttcatg
gcagcatgtcttgcatttggtaatgtagcctacgcagatgatgttaatgtaagtaattctaacgattatcttcacagtgatgga
agtaagcttttagatgactatggtaatcaggttagaatgactggtatagcttggtttggacttgagactccaaattactgttttc
atggcttgtgggctaataggcttgacaatattcttaatatagttgcagataatggatttaacactcttagagtgccattatctgt
tgaacttgtaaatcagtggcgtcaaggagtttatcctactccagattcaataaacgattatataagtcctgaattaaagggac
aaaatagccttcaaatacttgatgatgtcatagcttatagtaaaaaagttggcgtcaaagttatgctagatatgcacagaatt
gaaagtggtggacagacagctacttggtatacaagtaaatatactacagatgactatgaaaaatgttggcaatatcttgctg
atcgctataagaacgatgatacggttatagcagcagatatatttaacgagccacatggaaaagcttatagagcagaaacttc
tgctaagtggaatgatacaacagatgaagacaactggagatacgaagcagaaaaggttggtaaaaagattttagatataaa
tcctaagatgctcatagttgttgaaggagtagagacttaccctaaagagggaacagcagctggaagtacaaatcctgatgac
tactacggtggttggtggggaggaaatctaagaggtgttaaagattatcctgtagatttagccccatatGAATTC
-pGRNA_cel48A_E61Q
SEQ ID NO: 3
gagctcccgcggtgcggccgcgggccctactttttaacaaaatatattgataaaaataataatagtgggtataattaagttgt
tACTAAATGTAAAATGTTAGCgttttagagctagaaatagcaagttaaaataaggctagtccgttatcaacttgaaaa
agtggcaccgagtcggtgcttttttgtcgacgcggccgctctagaactagtgGATCcttgccgaaaaagggatctgacagta
aatccatcaggtttcattgttgcagatgaaaatgataaggatgtagatcatgcatcaacagatggaaaaataacaatcacag
gttcagcaccagttgttgtaaattcgtctgtaaatacttctagtgtaacttatgatccagcagcaccacaggatcaagctgtc
agtgttacacttaatggcaatacaatcacagatgtaaaggacgcaaacgctagtgttcttaaggctggaagtgattacacag
taacaagtgatggaattacactaagccaaagctatcttgctactctagcagcaggaacttacacatatacagttgattttagt
gcaggaaatgcaggtacatttactgttgttgttaagggaaaagcagtagtaaataaaactactttagcagtaggagctgcat
caggaaaagcaggagatactgttaaggtgcctgtaactataagtaaagtaacaacaccagtaggtttaatatgcatggaaat
agattatgatgcaagtaagtttactgttaaggatgtacttcctaatacagatcttgtaaaagatactgataactacagctttat
tgctaatacgacatcagcaggaaaaatcagtattacatttacagatccaacacttgagaaattcccaataagtgcagatgga
gttatagcaaatatagattttgttgtaaattcaggtgcagcaactggtgatagcgatttaacagtaaattcatcaggtttcatt
gttgcagatgaaagtgatacagatatagatcatgtatcaacaaatggaaaaataactgttgaataatcaatgacataatact
gccgccattataggatagcggcagtatactataaaattttaattaattatttttaaaggagatagaaaaatatgttaaagata
agtaagaattttaaaaaaataatggctgtagctcttacatctacagttatatttggttctctatctggattattaactaataaa
gttgcagctgctacaactacagattcatccttaaaagtagataatgcgtatactcaaagatttgaaacaatgtacaatAGAa
tgcacgatgctaataatggatatttcagtaaagatggtgttccatatcactcagttgaaacctttatggttgaggcacctgatt
atggtcatgaaactacaagtCAAgctttcagttattatatgtggcttgaagcaatgcagggaaaaatcacaggaaactttag
tggagtgaataccgcatgggatacagctgaaaagtatatgattccatcacaccaagatcaaccaggtatggatagatataac
gctagtagtccagcaacatattcaccagaatgggaagatccaagtaagtatccatctagaatggatcaaggagcagcaaaa
ggacaagatccaataagtgatgagcttaaatcagcatatggaacttcagatatgtatggaatgcattggttaatggatgttga
caactggtatggctttggaaatcatgaagatggaacatctaaaaatgtttatataaacacttatcaaagaggagaacaggaa
tctgtttttgaaacagtacctcaaccatgttgggatgctttcaaatatggtggaaaaaatggatatcttgacttattcacagga
gataatagctatgcaaaacaagctaaatacacagatgcaccagatgctgatgctcgtgcaatacaggcaacttatgaagca
gcacaagcagctaaagaggatggagtagatttaagctcaatcgtaggtaaagcttctaaaatgggagattacttaagatatg
ctatgtttgataaatattttagaaaaataggaaattcaactcaagcaggaaatggtaaagattcaatgcattatttactttctt
ggtattatgcttggggtggatctcaaaataatgattggtcttggaaaataggctgcagtcacagtcattttggatatcaaaatc
ctttaactgcatgggtactttcaactgatagtcaattcaaacctaaatcagcaactggtgcaactgactgggcaaagagttta
acaactcaggtagatttctatcaatggttacaatcttcagagggagctatagcaggtggtgctagtaactcaaatcatggtcg
ttatgaagcatggccagaaggtacagctacatttgatggaatgggatatcaagaagaaccagtttaccatgatccaggtagt
aacacatggtttggaatgcagtcatggtcaatgcagcgtatggctcaatactactatcaatcaaaagacccaaaagcaaaa
gctttacttgacaaatgggttaaatggattaaatctgtagttaaagtaaatccaaatggtgcaggaacatttgaagtaccatc
aaaattaagttggactggacaaccagatacatggacaggatcttatacaggaaatcctaacttacatgtaaatgttgattcat
atactactgatataggtacaagctcatcaactgcagatgcacttgcatactatgcagcagctacaggagataaggattcaca
agcactttcaaaaactatacttgatgatatctggaaaaactatcaggatgcaaagggtgtatcagcaccagaacaaatggac
tatagccgtgtattcaatcaagaagtttatatcccacaaggttggacaggaacaatgcctaatggagatgtaattaaatcag
gaaataaattcatagacatacgttcacaatataagaatgatcctgattatgctagagttaaatctgatgttgaagcaggaaa
gtcaagtacatttaactatcatcgtttctgggcagaaagtgagtatgctatagcaaatgcaaattatggaactttattcgctaa
tacagctacaccaggagatgtaaatggagacggcgtagttaatggaagagatcttatggagcttagacagtatttagcaggt
aagttagatgctagtaaaataaatttagctgcagcagatgttaataatgatggtgtagttaatggaagagatattatggaact
tactaaattaattgcaaaatagatcttataagaaaataacggaaggaagagtattcaatgaagaaaaaaggaataattaca
gcagtaatattgagtttactagttattggtacggtaggttgtaaatcaaacgacacaaagagtactgtatccgctaatcttgta
agcagtgaaaagtctaatttaaagataagcagtagtagcggaaaaacaggaagtaatgtggaggttaaagttaaggcaagt
aatgtagctaaaaagaatgttatttgctgcgattttaaaataaaatatgatgctagtaaattagacgtttctggtatttcacca
ggagaaattctaaaagatcctaaagataaccttgagtacaatgtggatagcaaaaatggtgttattacaattttatattcgta
ctcagataaaaatataggaaaagaattgatatcaaaaaatggggattttgtaactttaaacttgaaaataaaagatgatgct
aaaaagggaaaaactcaaataagctttaatggcaatcctgaattctatgataagaatcaaaaaggtgtgtcagttactacta
ataaaggtgaaatagaaattaaataggCcaggcatcaaataaaacgaaaggctcagtcgaaagactgggcctttcgtttta
tctgttgtttgtcggtgaacgctctcctgagtaggacaaatccgccgggagcggatttgaacgttgcgaagcaacggcccgga
gggtggcgggcaggacgcccgccataaactgccaggcatcaaattaagcagaaggccatcctgacggatggcctttttgcgt
ttcttagtagaatcttaatttatgggagaagtgtttgatatgaaaaaaaaagttttgcttaagtgctatttttgcatcgttcatg
gcagcatgtcttgcatttggtaatgtagcctacgcagatgatgttaatgtaagtaattctaacgattatcttcacagtgatgga
agtaagcttttagatgactatggtaatcaggttagaatgactggtatagcttggtttggacttgagactccaaattactgttttc
atggcttgtgggctaataggcttgacaatattcttaatatagttgcagataatggatttaacactcttagagtgccattatctgt
tgaacttgtaaatcagtggcgtcaaggagtttatcctactccagattcaataaacgattatataagtcctgaattaaagggac
aaaatagccttcaaatacttgatgatgtcatagcttatagtaaaaaagttggcgtcaaagttatgctagatatgcacagaatt
gaaagtggtggacagacagctacttggtatacaagtaaatatactacagatgactatgaaaaatgttggcaatatcttgctg
atcgctataagaacgatgatacggttatagcagcagatatatttaacgagccacatggaaaagcttatagagcagaaacttc
tgctaagtggaatgatacaacagatgaagacaactggagatacgaagcagaaaaggttggtaaaaagattttagatataaa
tcctaagatgctcatagttgttgaaggagtagagacttaccctaaagagggaacagcagctggaagtacaaatcctgatgac
tactacggtggttggtggggaggaaatctaagaggtgttaaagattatcctgtagatttagccccatatcagatccgtgagca
aaaggGAATTC
-pGRNA_Pthl-cel9X
SEQ ID NO: 4
gagctcccgcggtgcggccgcgggccctactttttaacaaaatatattgataaaaataataatagtgggtataattaagttgt
tacatatatattgacaccaatgttttagagctagaaatagcaagttaaaataaggctagtccgttatcaacttgaaaaagtgg
caccgagtcggtgcttttttgtcgacgcggccgctctagaactagtggatccaaagaagctggtttctttggattagttggtat
tgtgggtgctttggctgcacctgtagttgggaaagttgcagataaaaaaacaccaagatttgcagttggatttgctataatctt
ttctacaatagcttatgtttgtttttggggcatgggatataaaatatatggacttgtaattggagttattttgcttgatttaggaa
atcaaaccggacaagtttcaaatcaagcaagagttcaagctataagtgatcaagagagaagtcgtataaatactgtatttat
ggtttcatattttattggaggttcaattggatcatttgttgcagctgtattttggcaaaagtttggatggagtggtgtgtgtgcta
ttggtatgttttttcaaataattgcagttatatttcattattttatatacggaactaaatttagcattagaaataagtatggaca
ggtaaggtgatatataagtgtaaaattacaattttaaggttttagctatgaaaaagtagtctagaggcaggttgctttaaatat
aattttgtggtaaaatataactattaatataaagaatacctatttgtttggaaacaatgatgaatttctttaaattgggcacttg
agaaattttgagttagtagtgcaaccgaccaacgattaattaagctgtattaattgttggttttttgcttgtgtaaggaggtgtt
taaactattaaagtataaaaatgtaatttatgtattattgttcaatataatttacataatagggatataagatgtagagaggtt
aaataaaataatagccgactttttaacaaaatatattgataaaaataataatagtgggtataattaagttgttagagaaaac
gtataaattagggataaactatggaacttatgaaatagattgaaatggtttatctgttaccccgtagggcccagaatttaaaa
ggagggattaaaatgttaaggagaaaattactgtctatgatagttgctgcttctcttgtagttggagtaggattctctaatattt
gttatgcaaaaccaccagctccagatccaaacagcaatgtaggtacacatgatcttattagaaatagtacttttactgatgga
gttggattgccatggactgaggttgaaacagcacctgctcatggagattttgacatttcaggaggtacttataatataactgtt
acaaaaccaggaagtaatatctgggatgtacagtttagacatagaaatttacagcttaaagcaggacataaatatcatgtag
aatttacagtaacagcagataaggattgtgatatttatcctcaaatagctatgtctaaagatccatatactcaatattggcac
tatggaaactgggaaaatgtacacttaacagcaggacaagctaaaactgtaactgatgatttcactatgacaacaaatgatg
attctgctgaatttgcattccatatttccaatacaaacgataattcaaagcttccaataacatataaattcgataatatacatt
taacagatccacaatatacacaaccagcaataccagatgataatatttacgatgcggtaagggtaaaccaagtcggatatt
atcctaatcttgagaagattgcaacagtaacttctgattcttcaactccaataccatggaaacttcaagacagcacaggagc
agttgttgcatcaggacaaacaaaagtatttggacaagatcaggcatcaggagataatgttcatattatcgatttcacttcct
acaataaatccggtaaggactataaattagtagttagtggtgatactgatgaaaatccagcatacagtgttccttttaatatt
ggaagtgacttatattcacaacttaaacaagattctataaaatacttctatcacaacagaagtggtatagaaataaaaatgc
cttattgtggtgattcatgGaattc
-PGRNA_ΔnrpE
SEQ ID NO: 5
CTCGGGCCCTTGACAGCTAGCTCAGTCCTAGGTATAATACTAGTaatacaagtgatgataacgggttttag
agctagaaatagcaagttaaaataaggctagtccgttatcaacttgaaaaagtggcaccgagtcggtgcttttttGGATCC
atgttcttagtatattacctgtataacgattttctaagatttattttacgctccatattaacaaaaaatggattatacaactcta
aattttatcataataatacattatttccaattgcaaacataatagttattcacaagaaaaacccctctttctgtaagatgtaaa
cggttatttttacattaatgcttttaacaagcttttttgcttatttaacaacagttgacactttttatttacaaatattataattat
tatatactaaattacaaatttaataaaataatgcacttagggggaatattttATGTAAttctaggtataactaaaacactct
taccctattacaattatgtaatagggtatatctttctgaaatataatgctaattttgatattttcatttccacaatattttataac
tttatccatctagtatatacaaaattatcattacttttccaaatttttatttcatattacatcttatctacgaattacaatgcatt
atttaattttcatcatttgagacaattcttatatagtcatgctatataggtatataatggcaaactctgtactttaaagccgtgt
aaacgtttcttttcacttgaatatcactatagtaacttttttactatttatcaattagttgacatgtaactttttaaataatataa
ttatcacataattatgttatagatttaacaaaataacatacgaaaggaggaggcttttatgaaatgcaaattactttctactat
acttaccacaatcattttatccactttaggaagctttaacaatgtgGGCGCCGAATTC
-pGRNA_Pthl_cel5Y
SEQ ID NO: 6
ggagctcTTGACAGCTAGCTCAGTCCTAGGTATAATACTAGTatttctataagatatatgaagttttagagcta
gaaatagcaagttaaaataaggctagtccgttatcaacttgaaaaagtggcaccgagtcggtgcttttttggatccttatgaa
ggataaacgtttctctggttcaaaacgatgcataatgtagaagtagccgttttgtgcaagaaagagtgctatattttcatctat
aatagtttgcatatttgcaggtacaacaggcagtctaaagctatgctctcccaatattacacttgtatcgcattctgatcgact
acgtacaatgcattttgcgggaatcagttgaatatcttcgtaatcaaatacactttccataggtacacctctatatataaattt
taaggatagcttataatagtgtttgaaaatttatttatattatgcaaaattttagtgagattaatagtgaatgatgtagaaaag
aagtatgtaataataataacttactaaaatgttaaagtatacagccttacttaaaaaagtataaagtaattactagaaatatt
tataaacgttagctttttagcgtttatcttaataaatagaaggttgtaagatacaacaagaattaatagaaattccgACttttt
aacaaaatatattgatAaaaataataatagtgggtataattaagttgttagagaaaacgtataaattagggataaactatgg
aacttatgaaatagattgaaatggtttatctgttaccccgtagggcccagaatttaaaaggagggattaaaatgaggaacaa
aaaacgaataacatcgttagtaactggtttagctatgttattcacttgtgctgttggaaatacttctcttaaagtacatgccga
cgcacaatctatttacacaacgaaaggcgaaacgacaaagatttatgccagtgcttttacacaaaatactgatgactggact
tggatgagtatgggggatactgctagtctagtgtatcaggatgtgacaaatttcaatgcggtagatgctaatagtgcatttgca
aagtcaaattctactgccaactttggtataaacatttcggacggaaaccttgcagagggggattcaagtacattaaaatttca
tgttggtacggtcacagttaaagcaaatggctatgatgatcttgtaattaatttagataaagattattcagaagcgtatactgc
aacaaaatcttcctgggggcttacaggaaatactactcaaattttactaaatgattatttgccaaaagacacGaattc
-pMTL_JH16-xynB
SEQ ID NO: 9
caggataaaaaaattgtagataaattttataaaatagttttatctacaatttttttatcaggaaacagctatgaccgcggccat
ctatgcaacaaaagcagctattgaaaaagcaggttggacagttgatgaattagatttaatagaatcaaatgaagcttttgca
gctcaaagtttagcagtagcaaaagatttaaaatttgatatgaataaagtaaatgtaaatggaggagctattgcccttggtca
tccaattggagcatcaggtgcaagaatactcgttactcttgtacacgcaatgcaaaaaagagatgcaaaaaaaggcttagc
aactttatgtataggtggcggacaaggaacagcaatattgctagaaaagtgctagatcgattaagaaggagtgattacatga
acaaaaatataaaatattctcaaaactttttaacgagtgaaaaagtactcaaccaaataataaaacaattgaatttaaaaga
aaccgataccgtttacgaaattggaacaggtaaagggcatttaacgacgaaactggctaaaataagtaaacaggtaacgtc
tattgaattagacagtcatctattcaacttatcgtcagaaaaattaaaactgaatactcgtgtcactttaattcaccaagatat
tctacagtttcaattccctaacaaacagaggtataaaattgttgggagtattccttaccatttaagcacacaaattattaaaa
aagtggtttttgaaagccatgcgtctgacatctatctgattgttgaagaaggattctacaagcgtaccttggatattcaccgaa
cactagggttgctcttgcacactcaagtctcgattcagcaattgcttaagctgccagcggaatgctttcatcctaaaccaaaa
gtaaacagtgtcttaataaaacttacccgccataccacagatgttccagataaatattggaagctatatacgtactttgtttc
aaaatgggtcaatcgagaatatcgtcaactgtttactaaaaatcagtttcatcaagcaatgaaacacgccaaagtaaacaat
ttaagtaccgttacttatgagcaagtattgtctatttttaatagttatctattatttaacgggaggaaataaagcggccgcCTT
TAGAGAGGATGATACTATGAAAAAATTACTCACTGTAATTCTTATCTTGACACTTTTATCTATTCC
TTACTCTGTAAAATCTGCGAAAGCAGAAACTAATGTACGTGTCCCAGTTCTTCTATATCATGTTGT
TTCTACAAATCCAGACCCTAATAATCTTTATCAATTTAGTCTTACAGAATTCAAAAAGCATATGGA
TTATCTAAACGCTAATGGATATACGACACTTTCTATTGACCAATATTACAATATTATAAACAAAAAG
GCTCCTATGCCTAAGAAGCCAGTTATGCTTACCTTTGATGATTGTACTGAAGACTTCTATACAAAT
GTATATCCTATTTTAAGGAAATACCATATGAAAGCAGCCGAATTTGCAATCACAAATCTAATTGAT
ACCTATGGACATTTAACTACAAGTCAGCTTAAAACTGTTTTCTATAACGGAATTGATGTAGAGAAT
CACACTACAAATCACTTAGATTTAACTACTTTAACACATAACCAAAAGTATGCTGCAATCAATAATG
CAACTGCCAAAATTAAGTCTATAACCAATAAAGCTCCACTTTACTTGGCATACCCTTATGGAACAT
ATGATGCAGATAGTGTTTCAATCCTTAAAAGTTTAGGTTATAAAGCTGGTTTTTCCGTATCAAACG
TCTTAAGCACCGACACAAGTAACAAATATGGTTTACCTCGTATTGTTATTACAAATGGCGATACCT
TAAATGTATTTGAAAAAAAGCTTTTAAATGGTCATTAAAATTAATTATTAAATAAAAAAAGGAGTG
TTATTATGTTAAAATCAAAATTATCAAAAATATGTACAGGAGTCTTAGCTTTAGGTCTTGCCCTTT
CAATTTCAGGTGTAGGAACTTTTAAAGCTGCTATGTCACATAGCAAATTTGTAGGAAATATTATAG
CAGGAAGTATTCCTTCTAACTTTGATACCTATTGGAATCAAGTTACACCAGAAAATGCAACTAAGT
GGGGCGCAATTGAATATGGTCGTGGCAATTATAACTGGGGAAGCGCAGATCTTATTTATAATTAC
GCCAGAAGTAAAAACATGCCATTCAAATTTCATAATTTAGTATGGGGAAGTCAGCAGCCTACTTG
GTTGTCAAATCTTTCACCTCAAGATCAAAAATCTGAAGTATCAAAATGGATTGCAGCCGCAGGTCA
AAGATATTCTGGTTCAGCTTTTGTTGATGTTGTAAATGAACCACTGCATACTCAACCTTCTTACAA
AAATGCTTTAGGCGGAGATGGTTCCACCGGTTATGATTGGATTGTATGGTCTTATCAGCAGGCAA
GAAAAGCCTTCCCTAATTCAAAACTTTTAATTAATGAATATGGCATAATAGGCGATCCTAATGCAG
CAGCTAATTATGTTAAAATCATAAATGTTCTTAAAAGCAAAGGTTTAATTGATGGAATAGGAATTC
AATGTCACTATTTCAATATGGATAACGTTTCTGTAGGAACAATGAACTATGTTTTAAATATGTTAT
CTAATACAGGTTTACCAATATACGTATCAGAACTTGATATGACTGGCGATGACTCAACTCAGCTTG
CTAGATATCAACAAAAGTTCCCTGTTCTATATCAAAATCCTAATGTAAAAGGTATAACTTTATGGG
GATATATGCAAGGTCAAACTTGGAATAGTGGTACTTATTTAGTTAATTCAAATGGTACTGAACGT
CCAGCTCTTAAATGGTTAAGATCTTACTTAGCATCACATTAGctttgctagcaaagtattgttaaaaataact
ctgtagaattataaattagttctacagagttattttttaaaaaaattctaaacttatgtataaaaaatacgataagaatgtaga
attaaaactaaagacagttcaatttcttttagaataatttagttagtgtggtaaaaaaatgtcataatgatatttatgttgaaa
tttgtataaaattcagaaaatgaatatattttatcaattttcagtcatttgaaagattatgaggctaatgcagtactaggcgta
aattgaatttataattactatagcgataagaaatggcctaaaaacgtttgcagtaatgaaagaaccgtaaatattataaaaa
aaatcttaaaacagagttttatttataaaaatttaagatatataatttaaataacgtgttaaaatagtggaggaagtaatttga
atctgaatattaaaagaatgttaaaggttgtaactctttatgatgcaattattgctgcaatagtttcagtaatacttttgtttgc
tgctaattataagatttcgttaatagtgattatagggattttttcagcaatatttaatttttatttaagtaatttaacagctgatt
tcgtttttgtaaaaaaaatgggaaatacgtcacttatatttcttagttcaatttttagagtaatacttgttttttttataggtatta
ttctttataaaatatataaatattatttaatagcctacttaggaggatatagtgctcattttatagcccttataatttatgggtc
actagtaaataaacgatgaaaggaagtgattgaatggagctaggtgcaaagacagtattttcgatgaagcttggaagttaca
actttgctataacagaaactgtagtattacagtggattatcatggcagttataatattacttgcaatatttcttactaaaaatct
taagaaagtaccaaataggaaacaaagcgtaatagaaatgattgttaacttaataaatggattggtaaaagaaaatatggg
agagaaattcatgaatttcgttccaattatcggtactatggcagtgtttatacttttcttaaatttaacagggctagtaggtatc
gaaccagcaacaaaggatattagtgttacagcaggctttgctttagtaagtgcatttttaataaatgcaactgcaataaaaag
aaggcgcgccgcattcacttcttttctatataaatatgagcgaagcgaataagcgtcggaaaagcagcaaaaagtttcctttt
tgctgttggagcatgggggttcagggggtgcagtatctgacgtcaatgccgagcgaaagcgagccgaagggtagcatttacg
ttagataaccccctgatatgctccgacgctttatatagaaaagaagattcaactaggtaaaatcttaatataggttgagatga
taaggtttataaggaatttgtttgttctaatttttcactcattttgttctaatttcttttaacaaatgttcttttttttttagaacag
ttatgatatagttagaatagtttaaaataaggagtgagaaaaagatgaaagaaagatatggaacagtctataaaggctctca
gaggctcatagacgaagaaagtggagaagtcatagaggtagacaagttataccgtaaacaaacgtctggtaacttcgtaaa
ggcatatatagtgcaattaataagtatgttagatatgattggcggaaaaaaacttaaaatcgttaactatatcctagataatg
tccacttaagtaacaatacaatgatagctacaacaagagaaatagcaaaagctacaggaacaagtctacaaacagtaata
acaacacttaaaatcttagaagaaggaaatattataaaaagaaaaactggagtattaatgttaaaccctgaactactaatg
agaggcgacgaccaaaaacaaaaatacctcttactcgaatttgggaactttgagcaagaggcaaatgaaatagattgacct
cccaataacaccacgtagttattgggaggtcaatctatgaaatgcgattaagggccggccagtgggcaagttgaaaaattca
caaaaatgtggtataatatctttgttcattagagcgataaacttgaatttgagagggaacttagatggtatttgaaaaaattga
taaaaatagttggaacagaaaagagtattttgaccactactttgcaagtgtaccttgtacctacagcatgaccgttaaagtgg
atatcacacaaataaaggaaaagggaatgaaactatatcctgcaatgctttattatattgcaatgattgtaaaccgccattca
gagtttaggacggcaatcaatcaagatggtgaattggggatatatgatgagatgataccaagctatacaatatttcacaatg
atactgaaacattttccagcctttggactgagtgtaagtctgactttaaatcatttttagcagattatgaaagtgatacgcaac
ggtatggaaacaatcatagaatggaaggaaagccaaatgctccggaaaacatttttaatgtatctatgataccgtggtcaac
cttcgatggctttaatctgaatttgcagaaaggatatgattatttgattcctatttttactatggggaaatattataaagaagat
aacaaaattatacttcctttggcaattcaagttcatcacgcagtatgtgacggatttcacatttgccgttttgtaaacgaattg
caggaattgataaatagttaacttcaggtttgtctgtaactaaaaacaagtatttaagcaaaaacatcgtagaaatacggtgt
tttttgttaccctaagtttaaactcctttttgataatctcatgaccaaaatcccttaacgtgagttttcgttccactgagcgtca
gaccccgtagaaaagatcaaaggatcttcttgagatcctttttttctgcgcgtaatctgctgcttgcaaacaaaaaaaccacc
gctaccagcggtggtttgtttgccggatcaagagctaccaactctttttccgaaggtaactggcttcagcagagcgcagatac
caaatactgttcttctagtgtagccgtagttaggccaccacttcaagaactctgtagcaccgcctacatacctcgctctgcta
atcctgttaccagtggctgctgccagtggcgataagtcgtgtcttaccgggttggactcaagacgatagttaccggataaggc
gcagcggtcgggctgaacggggggttcgtgcacacagcccagcttggagcgaacgacctacaccgaactgagatacctaca
gcgtgagctatgagaaagcgccacgcttcccgaagggagaaaggcggacaggtatccggtaagcggcagggtcggaacag
gagagcgcacgagggagcttccagggggaaacgcctggtatctttatagtcctgtcgggtttcgccacctctgacttgagcgt
cgatttttgtgatgctcgtcaggggggcggagcctatggaaaaacgccagcaacgcggcctttttacggttcctggccttttgc
tggccttttgctcacatgttctttcctgcgttatcccctgattctgtggataaccgtattaccgcctttgagtgagctgataccg
ctcgccgcagccgaacgaccgagcgcagcgagtcagtgagcgaggaagcggaagagcgcccaatacgcagggccccctg
-pMTL_JH16-cipA-xynB
SEQ ID NO: 10
ctagcaaagtattgttaaaaataactctgtagaattataaattagttctacagagttattttttaaaaaaattctaaacttatg
tataaaaaatacgataagaatgtagaattaaaactaaagacagttcaatttcttttagaataatttagttagtgtggtaaaaa
aatgtcataatgatatttatgttgaaatttgtataaaattcagaaaatgaatatattttatcaattttcagtcatttgaaagatt
atgaggctaatgcagtactaggcgtaaattgaatttataattactatagcgataagaaatggcctaaaaacgtttgcagtaat
gaaagaaccgtaaatattataaaaaaaatcttaaaacagagttttatttataaaaatttaagatatataatttaaataacgtg
ttaaaatagtggaggaagtaatttgaatctgaatattaaaagaatgttaaaggttgtaactctttatgatgcaattattgctgc
aatagtttcagtaatacttttgtttgctgctaattataagatttcgttaatagtgattatagggattttttcagcaatatttaattt
ttatttaagtaatttaacagctgatttcgtttttgtaaaaaaaatgggaaatacgtcacttatatttcttagttcaatttttagag
taatacttgttttttttataggtattattctttataaaatatataaatattatttaatagcctacttaggaggatatagtgctcat
tttatagcccttataatttatgggtcactagtaaataaacgatgaaaggaagtgattgaatggagctaggtgcaaagacagta
ttttcgatgaagcttggaagttacaactttgctataacagaaactgtagtattacagtggattatcatggcagttataatatta
cttgcaatatttcttactaaaaatcttaagaaagtaccaaataggaaacaaagcgtaatagaaatgattgttaacttaataa
atggattggtaaaagaaaatatgggagagaaattcatgaatttcgttccaattatcggtactatggcagtgtttatacttttct
taaatttaacagggctagtaggtatcgaaccagcaacaaaggatattagtgttacagcaggctttgctttagtaagtgcattt
ttaataaatgcaactgcaataaaaagaaggcgcgccgcattcacttcttttctatataaatatgagcgaagcgaataagcgt
cggaaaagcagcaaaaagtttcctttttgctgttggagcatgggggttcagggggtgcagtatctgacgtcaatgccgagcga
aagcgagccgaagggtagcatttacgttagataaccccctgatatgctccgacgctttatatagaaaagaagattcaactag
gtaaaatcttaatataggttgagatgataaggtttataaggaatttgtttgttctaatttttcactcattttgttctaatttctttt
aacaaatgttcttttttttttagaacagttatgatatagttagaatagtttaaaataaggagtgagaaaaagatgaaagaaag
atatggaacagtctataaaggctctcagaggctcatagacgaagaaagtggagaagtcatagaggtagacaagttataccg
taaacaaacgtctggtaacttcgtaaaggcatatatagtgcaattaataagtatgttagatatgattggcggaaaaaaactta
aaatcgttaactatatcctagataatgtccacttaagtaacaatacaatgatagctacaacaagagaaatagcaaaagcta
caggaacaagtctacaaacagtaataacaacacttaaaatcttagaagaaggaaatattataaaaagaaaaactggagtat
taatgttaaaccctgaactactaatgagaggcgacgaccaaaaacaaaaatacctcttactcgaatttgggaactttgagca
agaggcaaatgaaatagattgacctcccaataacaccacgtagttattgggaggtcaatctatgaaatgcgattaagggccg
gccagtgggcaagttgaaaaattcacaaaaatgtggtataatatctttgttcattagagcgataaacttgaatttgagaggga
acttagatggtatttgaaaaaattgataaaaatagttggaacagaaaagagtattttgaccactactttgcaagtgtaccttg
tacctacagcatgaccgttaaagtggatatcacacaaataaaggaaaagggaatgaaactatatcctgcaatgctttattat
attgcaatgattgtaaaccgccattcagagtttaggacggcaatcaatcaagatggtgaattggggatatatgatgagatgat
accaagctatacaatatttcacaatgatactgaaacattttccagcctttggactgagtgtaagtctgactttaaatcattttt
agcagattatgaaagtgatacgcaacggtatggaaacaatcatagaatggaaggaaagccaaatgctccggaaaacatttt
taatgtatctatgataccgtggtcaaccttcgatggctttaatctgaatttgcagaaaggatatgattatttgattcctattttt
actatggggaaatattataaagaagataacaaaattatacttcctttggcaattcaagttcatcacgcagtatgtgacggatt
tcacatttgccgttttgtaaacgaattgcaggaattgataaatagttaacttcaggtttgtctgtaactaaaaacaagtattta
agcaaaaacatcgtagaaatacggtgttttttgttaccctaagtttaaactcctttttgataatctcatgaccaaaatccctta
acgtgagttttcgttccactgagcgtcagaccccgtagaaaagatcaaaggatcttcttgagatcctttttttctgcgcgtaat
ctgctgcttgcaaacaaaaaaaccaccgctaccagcggtggtttgtttgccggatcaagagctaccaactctttttccgaagg
taactggcttcagcagagcgcagataccaaatactgttcttctagtgtagccgtagttaggccaccacttcaagaactctgta
gcaccgcctacatacctcgctctgctaatcctgttaccagtggctgctgccagtggcgataagtcgtgtcttaccgggttgga
ctcaagacgatagttaccggataaggcgcagcggtcgggctgaacggggggttcgtgcacacagcccagcttggagcgaac
gacctacaccgaactgagatacctacagcgtgagctatgagaaagcgccacgcttcccgaagggagaaaggcggacaggt
atccggtaagcggcagggtcggaacaggagagcgcacgagggagcttccagggggaaacgcctggtatctttatagtcctgt
cgggtttcgccacctctgacttgagcgtcgatttttgtgatgctcgtcaggggggcggagcctatggaaaaacgccagcaacg
cggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaa
gagcgcccaatacgcagggccccctgcaggataaaaaaattgtagataaattttataaaatagttttatctacaatttttttat
caggaaacagctatgaccgcggccatctatgcaacaaaagcagctattgaaaaagcaggttggacagttgatgaattagat
ttaatagaatcaaatgaagcttttgcagctcaaagtttagcagtagcaaaagatttaaaatttgatatgaataaagtaaatgt
aaatggaggagctattgcccttggtcatccaattggagcatcaggtgcaagaatactcgttactcttgtacacgcaatgcaaa
aaagagatgcaaaaaaaggcttagcaactttatgtataggtggcggacaaggaacagcaatattgctagaaaagtgctaga
tcgattaagaaggagtgattacatgaacaaaaatataaaatattctcaaaactttttaacgagtgaaaaagtactcaaccaa
ataataaaacaattgaatttaaaagaaaccgataccgtttacgaaattggaacaggtaaagggcatttaacgacgaaactg
gctaaaataagtaaacaggtaacgtctattgaattagacagtcatctattcaacttatcgtcagaaaaattaaaactgaata
ctcgtgtcactttaattcaccaagatattctacagtttcaattccctaacaaacagaggtataaaattgttgggagtattcctt
accatttaagcacacaaattattaaaaaagtggtttttgaaagccatgcgtctgacatctatctgattgttgaagaaggattct
acaagcgtaccttggatattcaccgaacactagggttgctcttgcacactcaagtctcgattcagcaattgcttaagctgcca
gcggaatgctttcatcctaaaccaaaagtaaacagtgtcttaataaaacttacccgccataccacagatgttccagataaat
attggaagctatatacgtactttgtttcaaaatgggtcaatcgagaatatcgtcaactgtttactaaaaatcagtttcatcaag
caatgaaacacgccaaagtaaacaatttaagtaccgttacttatgagcaagtattgtctatttttaatagttatctattattta
acgggaggaaataaagcGGCCGCggcccagaatttaaaaggagggattaaaatgcgtaaaaagtctttagcatttttgtt
agcactaacaatgttggtgacattattaggggctcagcttacagcttttgcagcaggtactggcgtcGTTCAAATACAAtt
tgctgatacaaatactagtacaaccatgaatactattgctcctaaatttaaaatcacaaataatactggagcacctttagatt
taacaactttaaaattaagatactattttacagctgatggtactcaggatgaaaatttttggtgcgaccatgctggtatgctta
atggttataactaccaaacaattacaagtaatgtagtgggtacttttgtagctatggataatgcaacagctactgctgatcatt
atcttgagataagcttctcaaatggagcaggacaacttgatgcaggttcttcacttgaagttcaatgcagagttgcaaagaat
gactggagtaattatgatcaatcaaacgattattcatttacttctaatgcaagtgattttactgactgggataagataacaggt
tatgttaatggagatcttgtattcggaaatccaccagtagtagacccagttattactccaactactgctacatttgatacagct
aatccaactgatgtaaatgtagctatgcaattaaacggatttacattatcaggattaacagatgagaacggaacagctgttg
atgctgcaaattatacaatatcaggaagtaacttggtattaagcaaggcttatcttgcaaaattagcattaggcaaacatagc
tttaattttaattttgctaaaggcactacaactataagtaaaccattagcacttacagttacagatacagcaggtataacagt
agatcctgcaagtgtagtatttgataaagtagcacctcaagatgagaatgttgctcttaaattaaatggacatacacttggtg
atgtggtaggacctaaaggaaaccttgtaaaaggaacagattatactgtagcagatgatggaactgtaacatttagtaaagc
atatctttcaactcttccactaggtgatcagacaataacatttaaagctagtgatgattctactaaaacagcagcagtaacag
ttacagtgaagaattcaaatgctacaaatgttactgttggagatgttacaggagcaaaaaaaggagatactataaaggttcc
tgtaagtgtaagtacagtaaaaacaccaataggattaatagatatggaaataaactatgatccaacagagttaactgcaaa
agatgtagttcctacagatttagtaaaggatactgataactatagctttatagtaaatacatcaactccaggtaagattagta
ttacatttacagatccaactcttggaacttatccaataggtacagatggaatatttgcatatttagaattccttgtagctggtg
aaaaagcaggaaaatatgatttgaaagtaaacccaactacattaatacttgcagatgaaaatgataatgatatagattgtaa
cccaccaaaggatggaagtgtaactgttataggtactccagttgtaactccatctcaaataaatgttgaacaaggttcagca
actgatcagccagttaaaatagatttaaatggtaacacacttaaagatgttgttgatcaaagtggtaagactcttgttcaagg
aacggattatacagtaacagatactggaatcacattaagtcaatcttatcttgcaggtttagccttaggtcaatacactctaa
cacttgattttaatggtggaggagcatcacagacaataactattaatgttgtaaagaatgaaactgtaaaattgtcagttgga
acagtatcaggaaatccaggggatactgtaaaggtgcctgtaactataagccaggtatcaacaccagtaggtttaatatgta
tggatataagctacgatgcaagtaagtttactgttaaggatgtacttcctaatacagatcttgtaaaggatactgataactac
agctttatagtaaatacatcaactccaggtaagattagtattacatttacagatccaacacttgcaaactatccaataagtgt
agacggaattcttgcatacttagactttattataaattcaaatgcaacagcaggagatagtgctttaacagtagatccagcta
cattaatagttgcagatgaaaatgataaagatataaaagatgcagcttctaatggaaaaataacagttacaggttcagcacc
agttgttcaaagctcagtagtaaatacttcaagtgtaacttatgatcaaaatgcaccacaggatcaagctgttagtattacttt
caatggcaatacagttaaggatgtaaaggacgcaagtggcaatacacttaaggctggaagtgactacacagcaacaagtga
tggaattacacttagccaaagttatcttgctactttagcagcaggaacttacacatatacaattgattttagtgcaggaaatg
cagggacatttactgttgttgttaaaggaaaaacagtagtaggtagtgcaactactttagcagtaggaactgtatcaggaaa
agcaggagatactgtaaaggtacctgtaactatcagtaaagtaacaacaccagtaggtttaatatgtgcagaaatagactat
gatgcaagcaagtttactgttaaggatgtacttcctaatacagatcttgtaaaggatactgataactacagctttatagtaaat
acatcaactccaggtaagattagtattacatttacagatccaacacttgcaaactatccaataagtgcagatggaattcttgc
atacttagactttatcataaactcaaatgcaacagcaggggatagtgctttaacagtaaatccatcaggatttatcattgcag
atgaaaatgataaagatatacaggatgcagcttctaacggaaaaataacagttacaggttcaacaccagttgctgaaaattc
agtagtaaatacttcaagtgtaacttatgatcaaaatgcaccacaggatcaagctgttagcattaccttaaatggtaatacaa
ttacagatgtaaaagatgcaagtggtaatacacttaaggctggaagtgattacacagtaacaagtgatggaattacacttag
ccaaagctaccttgctactttagcagcaggaacttacacatatacagttgattttagtgcaggaaatgcaggaacatttactg
ttgttgttaaagcaaaaacagtagtaagtagtgcaactactttagcagtaggaacagtatcaggaaaagcaggagatactgt
aaaggtacctgtaactataagtaaagtaacaacaccagtagggttaatatgtgcagaaatagattatgatgcaagcaagttt
actgttaaggatgtacttcctaatacagatcttgtaaaagatactgataactacagctttatagtaaatactgcaacagcagg
aaaaatcagtattacattcacagatccaacacttgagaaattcccaataagtgcagatggaattcttgcatacttagacttta
tcataaactcaaatgcaacagcaggggatagtgctttaacagtaaatccatcaggtttcattgttgcagatgaaaatgataa
ggatgtagatcatgcatcaacagatggaaaaataacaatcacaggttcagcaccagttgttgtaaattcgtctgtaaatactt
ctagtgtaacttatgatccagcagcaccacaggatcaagctgtcagtgttacacttaatggcaatacaatcacagatgtaaa
ggacgcaaacgctagtgttcttaaggctggaagtgattacacagtaacaagtgatggaattacactaagccaaagctatctt
gctactctagcagcaggaacttacacatatacagttgattttagtgcaggaaatgcaggtacatttactgttgttgttaaggga
aaagcagtagtaaataaaactactttagcagtaggagctgcatcaggaaaagcaggagatactgttaaggtgcctgtaacta
taagtaaagtaacaacaccagtaggtttaatatgcatggaaatagattatgatgcaagtaagtttactgttaaggatgtactt
cctaatacagatcttgtaaaagatactgataactacagctttattgctaatacgacatcagcaggaaaaatcagtattacatt
tacagatccaacacttgagaaattcccaataagtgcagatggagttatagcaaatatagattttgttgtaaattcaggtgcag
caactggtgatagcgatttaacagtaaattcatcaggtttcattgttgcagatgaaagtgatacagatatagatcatgtatca
acaaatggaaaaataactgttgaataaGCggccgcCTTTAGAGAGGATGATACTATGAAAAAATTACTCACT
GTAATTCTTATCTTGACACTTTTATCTATTCCTTACTCTGTAAAATCTGCGAAAGCAGAAACTAAT
GTACGTGTCCCAGTTCTTCTATATCATGTTGTTTCTACAAATCCAGACCCTAATAATCTTTATCAA
TTTAGTCTTACAGAATTCAAAAAGCATATGGATTATCTAAACGCTAATGGATATACGACACTTTCT
ATTGACCAATATTACAATATTATAAACAAAAAGGCTCCTATGCCTAAGAAGCCAGTTATGCTTACC
TTTGATGATTGTACTGAAGACTTCTATACAAATGTATATCCTATTTTAAGGAAATACCATATGAAA
GCAGCCGAATTTGCAATCACAAATCTAATTGATACCTATGGACATTTAACTACAAGTCAGCTTAAA
ACTGTTTTCTATAACGGAATTGATGTAGAGAATCACACTACAAATCACTTAGATTTAACTACTTTA
ACACATAACCAAAAGTATGCTGCAATCAATAATGCAACTGCCAAAATTAAGTCTATAACCAATAAA
GCTCCACTTTACTTGGCATACCCTTATGGAACATATGATGCAGATAGTGTTTCAATCCTTAAAAGT
TTAGGTTATAAAGCTGGTTTTTCCGTATCAAACGTCTTAAGCACCGACACAAGTAACAAATATGGT
TTACCTCGTATTGTTATTACAAATGGCGATACCTTAAATGTATTTGAAAAAAAGCTTTTAAATGGT
CATTAAAATTAATTATTAAATAAAAAAAGGAGTGTTATTATGTTAAAATCAAAATTATCAAAAATAT
GTACAGGAGTCTTAGCTTTAGGTCTTGCCCTTTCAATTTCAGGTGTAGGAACTTTTAAAGCTGCT
ATGTCACATAGCAAATTTGTAGGAAATATTATAGCAGGAAGTATTCCTTCTAACTTTGATACCTAT
TGGAATCAAGTTACACCAGAAAATGCAACTAAGTGGGGCGCAATTGAATATGGTCGTGGCAATTA
TAACTGGGGAAGCGCAGATCTTATTTATAATTACGCCAGAAGTAAAAACATGCCATTCAAATTTCA
TAATTTAGTATGGGGAAGTCAGCAGCCTACTTGGTTGTCAAATCTTTCACCTCAAGATCAAAAATC
TGAAGTATCAAAATGGATTGCAGCCGCAGGTCAAAGATATTCTGGTTCAGCTTTTGTTGATGTTG
TAAATGAACCACTGCATACTCAACCTTCTTACAAAAATGCTTTAGGCGGAGATGGTTCCACCGGTT
ATGATTGGATTGTATGGTCTTATCAGCAGGCAAGAAAAGCCTTCCCTAATTCAAAACTTTTAATTA
ATGAATATGGCATAATAGGCGATCCTAATGCAGCAGCTAATTATGTTAAAATCATAAATGTTCTTA
AAAGCAAAGGTTTAATTGATGGAATAGGAATTCAATGTCACTATTTCAATATGGATAACGTTTCTG
TAGGAACAATGAACTATGTTTTAAATATGTTATCTAATACAGGTTTACCAATATACGTATCAGAAC
TTGATATGACTGGCGATGACTCAACTCAGCTTGCTAGATATCAACAAAAGTTCCCTGTTCTATATC
AAAATCCTAATGTAAAAGGTATAACTTTATGGGGATATATGCAAGGTCAAACTTGGAATAGTGGT
ACTTATTTAGTTAATTCAAATGGTACTGAACGTCCAGCTCTTAAATGGTTAAGATCTTACTTAGCA
TCACATTAGctttg

### BIBLIOGRAPHIC REFERENCES

Abdou L. et al. ‘Transcriptional Regulation of the Clostridium cellulolyticum cip-cel Operon: a Complex Mechanism Involving a Catabolite-Responsive Element. JOURNAL OF BACTERIOLOGY, March 2008, p. 1499-1506.
Bayer, E A, and R Lamed. 1986. ‘Ultrastructure of the Cell Surface Cellulosome of Clostridium Thermocellum and Its Interaction with Cellulose.’ Journal of Bacteriology 167 (3): 828-36.
Bayer, Edward A., Yuval Shoham, and Raphael Lamed. 2002. ‘The Cellulosome’. In Glycomicrobiology, edited by Ron J. Doyle, 387-439. Boston, MA: Springer U S. https://doi.org/10.1007/0-306-46821-2_14.
Compere, A. L., and W. L. Griffith. 1979. ‘Evaluation of Substrates for Butanol Production’. Dev. Microbiol.; Ind. (United States) 20 (January). https://www.osti.gov/biblio/6510996-evaluation-substrates-butanol-production.
Dassa, Bareket, Ilya Borovok, Vincent Lombard, Bernard Henrissat, Raphael Lamed, Edward A. Bayer, and Sarah Moraïs. 2017. “Pan-Cellulosomics of Mesophilic Clostridia: Variations on a Theme” Microorganisms 5, no. 4:74 https://doi.org/10.3390/microorganisms5040074.
Desvaux Mickaël, Clostridium cellulolyticum: model organism of mesophilic cellulolytic clostridia, FEMS Microbiology Reviews, Volume 29, Issue 4, September 2005, Pages 741-764, https://doi.org/10.1016/j.femsre.2004.11.003.
Doi, Roy H., Marc Goldstein, Seiichi Hashida, Jae-Seon Park, and Masahiro Takagi. 1994. ‘The Clostridium Cellulovorans Cellulosome’. Critical Reviews in Microbiology 20 (2): 87-93. https://doi.org/10.3109/10408419409113548.
Dunning, J. W., and E. C. Lathrop. 1945. ‘Saccharification of Agricultural Residues’. Industrial & Engineering Chemistry 37 (1): 24-29. https://doi.org/10.1021/ie50421a006.
Dusséaux Simon, Croux Christian, Soucaille Philippe, Meynial-Salles Isabelle. ‘Metabolic engineering of Clostridium acetobutylicum ATCC 824 for the high-yield production of a biofuel composed of an isopropanol/butanol/ethanol mixture’. Metabolic Engineering, Volume 18, 2013, Pages 1-8, https://doi.org/10.1016/j.ymben.2013.03.003.
EP2436698A1
Ehsaan, M., Kuit, W., Zhang, Y. et al. Mutant generation by allelic exchange and genome resequencing of the biobutanol organism Clostridium acetobutylicum ATCC 824. Biotechnol Biofuels 9, 4 (2016). https://doi.org/10.1186/s13068-015-0410-0
Foulquier C. et al. ‘Molecular Characterization of the Missing Electron Pathways for Butanol Synthesis in Clostridium Acetobutylicum’. Nature Communications | (2022) 13:4691.
Gal, L, S Pages, C Gaudin, A Belaich, C Reverbel-Leroy, C Tardif, and J P Belaich. 1997. ‘Characterization of the Cellulolytic Complex (Cellulosome) Produced by Clostridium Cellulolyticum’. Applied and Environmental Microbiology 63 (3): 903-9. https://doi.org/10.1128/aem.63.3.903-909.1997.
Heap J T et L. The ClosTron: Mutagenesis in Clostridium refined and streamlined, Journal of Microbiological Methods, Volume 80, Issue 1, 2010, Pages 49-55.
Kakiuchi, M., A. Isui, K. Suzuki, T. Fujino, E. Fujino, T. Kimura, S. Karita, K. Sakka, and K. Ohmiya. 1998. ‘Cloning and DNA Sequencing of the Genes Encoding Clostridium Josui Scaffolding Protein CipA and Cellulase CelD and Identification of Their Gene Products as Major Components of the Cellulosome’. Journal of Bacteriology 180 (16): 4303-8.
Lee, Song F., Cecil W. Forsberg, and L. N. Gibbins. 1985. ‘Cellulolytic Activity of Clostridium Acetobutylicum’. Applied and Environmental Microbiology 50 (2): 220-28.
Lehmann, Dörte, and Tina Lütke-Eversloh. 2011. ‘Switching Clostridium Acetobutylicum to an Ethanol Producer by Disruption of the Butyrate/Butanol Fermentative Pathway’. Metabolic Engineering 13 (5): 464-73. https://doi.org/10.1016/j.ymben.2011.04.006.
Mingardon F. et al. “The Issue of Secretion in Heterologous Expression of Clostridium cellulolyticum Cellulase-Encoding Genes in Clostridium acetobutylicum ATCC 824”. APPLIED AND ENVIRONMENTAL MICROBIOLOGY, May 2011, p. 2831-2838. doi:10.1128/AEM.03012-10.
Nguyen, Ngoc-Phuong-Thao, Celine Raynaud, Isabelle Meynial-Salles, and Philippe Soucaille. 2018. ‘Reviving the Weizmann Process for Commercial n-Butanol Production’. Nature Communications 9 (1): 1-8. https://doi.org/10.1038/s41467-018-05661-z.
Nölling, Jörk, Gary Breton, Marina V. Omelchenko, Kira S. Makarova, Qiandong Zeng, Rene Gibson, Hong Mei Lee, et al. 2001. ‘Genome Sequence and Comparative Analysis of the Solvent-Producing Bacterium Clostridium Acetobutylicum’. Journal of Bacteriology 183 (16): 4823-38. https://doi.org/10.1128/JB.183.16.4823-4838.2001.
O'Brien R. W. and Morris J. G. ‘Oxygen and the Growth and Metabolism of Clostridium acetobu tylicum”. Journal of General Microbiology (1971), 68, 307-318.
Oultram J. D., M. Loughlin, T-J. Swinfield, J. K. Brehm, D. E. Thompson, N. P. Minton, Introduction of plasmids into whole cells of Clostridium acetobutylicum by electroporation, FEMS Microbiology Letters, Volume 56, Issue 1, November 1988, Pages 83-88, https://doi.org/10.1111/j.1574-6968.1988.tb03154.x
Reverbel-Leroy, C., S. Pages, A. Belaich, J. P. Belaich, and C. Tardif. 1997. ‘The Processive Endocellulase CelF, a Major Component of the Clostridium Cellulolyticum Cellulosome: Purification and Characterization of the Recombinant Form.’ Journal of Bacteriology 179 (1): 46-52. https://doi.org/10.1128/jb.179.1.46-52.1997.
Sabathé, Fabrice, Anne Belaïch, and Philippe Soucaille. 2002. ‘Characterization of the Cellulolytic Complex (Cellulosome) of Clostridium Acetobutylicum’. FEMS Microbiology Letters 217 (1): 15-22. https://doi.org/10.1111/j.1574-6968.2002.tb11450.x.
Soni, B. K., Soucaille, P. & Goma, G. Continuous acetone-butanol fermentation: a global approach for the improvement in the solvent productivity in synthetic medium. Appl Microbiol Biotechnol 25, 317-321 (1987). https://doi.org/10.1007/BF00252540 Sticheblykina, N. A., and Nakhamanovich, B. M. 1959. ‘Fermentation of Pentoses of Corn Cob Hydrolyzates by Clostridium Acetobutylicum’. Mikrobiologiya 28:91-96.
Sudha Rani, K., M. V. Swamy, and G. Seenayya. 1998. ‘Production of Ethanol from Various Pure and Natural Cellulosic Biomass by Clostridium Thermocellum Strains SS21 and SS22’. Process Biochemistry 33 (4): 435-40. https://doi.org/10.1016/S0032-9592(97)00095-2.
Tamaru Y, Karita S, Ibrahim A, Chan H, Doi R H. A large gene cluster for the Clostridium cellulovorans cellulosome. J Bacteriol. 2000 October; 182(20):5906-10. doi: 10.1128/JB.182.20.5906-5910.2000.
TAO XUANYU ET AL: “Precise promoter integration improves cellulose bioconversion and thermotolerance in Clostridium cellulolyticum”, METABOLIC ENGINEERING, vol. 60, 1 Jul. 2020 (2020-07-01), pages 110-118, ISSN: 1096-7176, DOI: 10.1016/j.ymben.2020.03.013.
US20170240869A1
WO2008040387
Wilding-Steele, Tom, Quentin Ramette, Paul Jacottin, and Philippe Soucaille. 2021. ‘Improved CRISPR/Cas9 Tools for the Rapid Metabolic Engineering of Clostridium Acetobutylicum’. International Journal of Molecular Sciences 22 (7): (3704. https://doi.org/10.3390/ijms22073704.
Xu, C., Huang, R., Teng, L. et al. Cellulosome stoichiometry in Clostridium cellulolyticum is regulated by selective RNA processing and stabilization. Nat Commun 6, 6900 (2015). https://doi.org/10.1038/ncomms7900.
Yutin N et al. “A genomic update on clostridial phylogeny: Gram-negative spore formers and other misplaced clostridia”. Genomics update. Vol. 15, Issue 10, October 2013, pages 2631-2641, https://doi.org/10.1111/1462-2920.12173.
Zhao, Lei, Zhong-Fang Sun, Cheng-Cheng Zhang, Jun Nan, Nan-Qi Ren, Duu-Jong Lee, and Chuan Chen. 2022. ‘Advances in Pretreatment of Lignocellulosic Biomass for Bioenergy Production: Challenges and Perspectives’. Bioresource Technology 343 (January): 126123. https://doi.org/10.1016/j.biortech.2021.126123.

1.-15. (canceled)

16. A genetically modified Clostridium acetobutylicum able to grow on lignocellulosic biomass, in which the cip-cel operon is overexpressed, the expression level of each gene of the cip-cel operon in the genetically modified Clostridium acetobutylicum being higher than its expression level in a corresponding non-genetically modified Clostridium acetobutylicum.

17. The genetically modified Clostridium acetobutylicum according to claim 16, wherein:
a) the native promoter of the cip-cel operon has been genetically modified, or
b) an additional copy of the cip-cel operon has been inserted in the Clostridium acetobutylicum genome under the control of a promoter stronger than the native promoter of the cip-cel operon.

18. The genetically modified Clostridium acetobutylicum according to claim 17, wherein:
a) the native promoter of the cip-cel operon has been replaced by a stronger promoter, or
b) the native promoter of the cip-cel operon has been mutated.

19. The genetically modified Clostridium acetobutylicum according to claim 18, wherein the stronger promoter is selected from Clostridium acetobutylicum thlA gene promoter, ptb gene, crt gene, and the gapC gene.

20. The genetically modified Clostridium acetobutylicum according to claim 16, wherein the native promoter of the cip-cel operon has been replaced by the promoter of Clostridium acetobutylicum thlA gene.

21. The genetically modified Clostridium acetobutylicum according to claim 16, in which the cel48A gene encodes the native Cel48A protein of Clostridium acetobutylicum of amino acid sequence WP_010964229.1.

22. The genetically modified Clostridium acetobutylicum according to claim 16, which has been further genetically modified:
a) so that the cel9X gene is overexpressed, its expression level in the genetically modified Clostridium acetobutylicum being higher than its expression level in a corresponding non-genetically modified Clostridium acetobutylicum;
b) so that the cel5Y gene is overexpressed, its expression level in the genetically modified Clostridium acetobutylicum being higher than its expression level in a corresponding non-genetically modified Clostridium acetobutylicum;
c) so that the xynB gene is overexpressed, its expression level in the genetically modified Clostridium acetobutylicum being higher than its expression level in a corresponding non-genetically modified Clostridium acetobutylicum;
d) so that the cipA gene is further overexpressed compared to other genes of the cip-cel operon;
e) by deletion of a gene encoding an extracellular protease; or
f) any combination of a) to e).

23. The genetically modified Clostridium acetobutylicum according to claim 22, wherein:
a) the native promoter of the cel9X gene has been genetically modified, or
b) an additional copy of the cel9X gene has been inserted in the Clostridium acetobutylicum genome under the control of a promoter stronger than the native promoter of the cel9X gene.

24. The genetically modified Clostridium acetobutylicum according to claim 23, wherein the native promoter of the cel9X gene has been replaced by a stronger promoter selected from Clostridium acetobutylicum thlA gene promoter, ptb gene promoter, crt gene promoter, and gapC gene promoter.

25. The genetically modified Clostridium acetobutylicum according to claim 22, wherein:
a) the native promoter of the cel5Y gene has been genetically modified, or
b) an additional copy of the cel5Y gene has been inserted in the Clostridium acetobutylicum genome under the control of a promoter stronger than the native promoter of the cel5Y gene.

26. The genetically modified Clostridium acetobutylicum according to claim 25, wherein the native promoter of the cel5Y gene has been replaced by a stronger promoter selected from Clostridium acetobutylicum thlA gene promoter, ptb gene promoter, crt gene promoter and the gapC gene promoter.

27. The genetically modified Clostridium acetobutylicum according to claim 22, wherein:
a) an additional copy of the xynB gene has been inserted in the Clostridium acetobutylicum genome under the control of a promoter stronger than the native promoter of the xynB gene,
b) the native promoter of the xynB gene has been genetically modified.

28. The genetically modified Clostridium acetobutylicum according to claim 27, wherein an additional copy of the xynB gene has been inserted in the Clostridium acetobutylicum genome under the control of a promoter stronger than the native promoter of the xynB gene selected from Clostridium acetobutylicum thlA gene promoter, ptb gene promoter, crt gene promoter, and gapC gene promoter.

29. The genetically modified Clostridium acetobutylicum according to claim 22, wherein an additional copy of the cipA gene has been inserted in the Clostridium acetobutylicum genome under the control of a promoter stronger than the native promoter of the cipA gene.

30. The genetically modified Clostridium acetobutylicum according to claim 22, wherein the extracellular protease is selected from nrpE, CA_C0746, CA_C0625, and CA_C2695.

31. The genetically modified Clostridium acetobutylicum according to claim 16, which is selected from:
a) a genetically modified Clostridium acetobutylicum in which the native promoter of the cip-cel operon has been replaced by the promoter of Clostridium acetobutylicum thlA gene,
b) a genetically modified Clostridium acetobutylicum in which the native promoter of the cip-cel operon has been replaced by the promoter of Clostridium acetobutylicum thlA gene and the native promoter of the cel9X gene has been replaced by the promoter of Clostridium acetobutylicum thlA gene,
c) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and the native promoter of the cel5Y gene has been replaced by the promoter of Clostridium acetobutylicum thlA gene,
d) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and an additional copy of the xynB gene has been inserted in the Clostridium acetobutylicum genome under the control of the promoter of Clostridium acetobutylicum thlA gene,
e) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, and the nrpE gene has been partially or completely deleted,
f) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, and the nrpE gene has been partially or completely deleted,
g) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, an additional copy of the xynB gene has been inserted in the Clostridium acetobutylicum genome under the control of the promoter of Clostridium acetobutylicum thlA gene, and the nrpE gene has been partially or completely deleted, and
h) a genetically modified Clostridium acetobutylicum in which the native promoter of the cip-cel operon has been replaced by the promoter of Clostridium acetobutylicum thlA gene, the native promoter of the cel9X gene has been replaced by the promoter of Clostridium acetobutylicum thlA gene, the native promoter of the cel5Y gene has been replaced by the promoter of Clostridium acetobutylicum thlA gene, an additional copy of the xynB gene has been inserted in the Clostridium acetobutylicum genome under the control of the promoter of Clostridium acetobutylicum thlA gene, an additional copy of the cipA gene has been inserted in the Clostridium acetobutylicum genome under the control of the promoter of Clostridium acetobutylicum thlA gene and the nrpE gene has been partially or completely deleted.

32. The genetically modified Clostridium acetobutylicum according to claim 16, which has been further optimized for:
a) ethanol production by inactivation of the IdhA gene, the thlA gene, and the hydA gene,
b) butanol production by inactivation of the ptb-buk operon, replacement of the Clostridium acetobutylicum thlA gene by Escherichia coli atoB gene, replacement of the Clostridium acetobutylicum hbd gene by Clostridium kluyveri hbd1 gene, and overexpression of CA_C0764 gene, its expression level being higher than its expression level in a corresponding non-genetically modified Clostridium acetobutylicum; or
c) lactate production by inactivation of the thlA gene and the hydA gene, followed by curing of the megaplasmid.

33. The genetically modified Clostridium acetobutylicum according to claim 22, which has been further optimized for:
a) ethanol production by inactivation of the IdhA gene, the thlA gene, and the hydA gene,
b) butanol production by inactivation of the ptb-buk operon, replacement of the Clostridium acetobutylicum thlA gene by Escherichia coli atoB gene, replacement of the Clostridium acetobutylicum hbd gene by Clostridium kluyveri hbd1 gene, and overexpression of CA_C0764 gene, its expression level being higher than its expression level in a corresponding non-genetically modified Clostridium acetobutylicum; or
c) lactate production by inactivation of the thlA gene and the hydA gene, followed by curing of the megaplasmid.

34. The genetically modified Clostridium acetobutylicum according to claim 31, which has been further optimized for:
a) ethanol production by inactivation of the IdhA gene, the thlA gene, and the hydA gene,
b) butanol production by inactivation of the ptb-buk operon, replacement of the Clostridium acetobutylicum thlA gene by Escherichia coli atoB gene, replacement of the Clostridium acetobutylicum hbd gene by Clostridium kluyveri hbd1 gene, and overexpression of CA_C0764 gene, its expression level being higher than its expression level in a corresponding non-genetically modified Clostridium acetobutylicum; or
c) lactate production by inactivation of the thlA gene and the hydA gene, followed by curing of the megaplasmid.

35. A method for the production of a targeted bulk chemical from lignocellulosic biomass, comprising culturing a genetically modified Clostridium acetobutylicum according to claim 16, on an appropriate culture medium comprising lignocellulosic biomass as main source of carbon, and recovering the targeted bulk chemical from the culture medium.
